(19) United States
(12) Patent Application Publication
(10) Pub. No.: US 2017/0027679 A1
(43) Pub. Date: Feb. 2, 2017

(54) MEDICAL DEVICE WITH ANTI ADHESIVE PROPERTY

(71) Applicant: Allergan, Inc., Irvine, CA (US)

(72) Inventors: Monica A. Serban, Melrose, MA (US); Susan E. Burke, Wilbraham, MA (US); William A. Daunch, Cary, NC (US); Vinit Patel, Waltham, MA (US); Bryan W. Jones, Woburn, MA (US)

(21) Appl. No.: 15/293,148

(22) Filed: Oct. 13, 2016

Related U.S. Application Data
(63) Continuation of application No. 14/458,549, filed on Aug. 13, 2014.
Application No. 14/458,549 is a continuation-in-part of application No. 13/973,818, filed on Aug. 22, 2013, now abandoned.

(51) Int. Cl. A61F 2/00; D04B 21/20; A61L 31/10; D04B 21/16; A61L 31/04; A61L 31/14

(52) U.S. Cl. CPC A61F 2/0063; A61F 2/0077; A61L 31/047; A61L 31/146; A61L 31/10; D04B 21/16; D04B 21/20; A61F 2002/009; A61F 2002/0068

(57) ABSTRACT

Laminate or knitted medical devices and methods using such devices to support soft tissues and/or to reduce formation of post-operative adhesions. The medical devices can comprise a layer of a knitted silk mesh to which has been fused a water soluble or insoluble silk film or silk sponge, and/or a layer of a knitted silk mesh which was co-knitted with one, two or three layers of silk or non-silk fabric.

### CROSS REFERENCE

This application is a continuation of U.S. patent application Ser. No. 14/458,549, filed on Aug. 13, 2014, which is a continuation-in-part of U.S. patent application Ser. No. 13/973,818, filed Aug. 22, 2013, the entire content of each of which is incorporated herein by reference.

### BACKGROUND

The present invention relates to implantable medical devices made entirely or partially of silk, including silk medical devices with at least one surface (i.e. a top surface or a bottom surface of the silk medical device) made or prepared so that, after in vivo implantation of the silk medical device (such as implantation in conjunction with a medical or surgical procedure, such as an abdominal procedure, such as a hernia repair procedure) adhesion or attachment of a tissue (such as a human abdominal, bowel or intestinal tissue) to that surface or surfaces of the silk medical device is prevented, substantially prevented, discouraged and/or not facilitated (hence an “anti-adhesive” surface). In particular the present invention relates to single layer and multi-laminate, anti-adhesive surface silk based devices comprising one or more of a silk film, a silk sponge, and a knitted silk fiber or fabric as well as methods for making and using, for example in abdominal surgery. The devices can be combined with or coated with a hyaluronic acid or other macromolecule (such as for example dextran, heparin and sulphates thereof)

Silk is a natural (non-synthetic) protein that can be processed into high strength fibroin fibers with mechanical properties similar to or better than many of synthetic high performance fibers. Silk is stable at physiological temperatures in a wide range of pH, and is insoluble in most aqueous and organic solvents. As a protein, unlike the case with most if not all synthetic polymers, the degradation products (e.g. peptides, amino acids) of silk are biocompatible. Silk is non-mammalian derived and carries far less bioburden than other comparable natural biomaterials (e.g. bovine or porcine derived collagen). Silk, as the term is generally known in the art, means a filamentous fiber product secreted by an organism such as a silkworm or spider. Silks can be made by certain insects such as for example Bombyx mori silkworms, and Nephilia clavipes spiders. There are many variants of natural silk. Fibroin is produced and secreted by a silkworm's two silk glands. As fibroin leaves the glands it is coated with sericin a glue-like substance. Spider silk is produced as a single filament lacking the immunogenic protein sericin.

Silk has been used in biomedical applications. The Bombyx mori species of silkworm produces a silk fiber (a “bave”) and uses the fiber to build its cocoon. The bave as produced include two fibroin filaments or broins which are surrounded with a coating of the gummy, antigenic protein sericin. Silk fibers harvested for making textiles, sutures and clothing are not sericin extracted or are sericin depleted or only to a minor extent and typically the silk remains at least 10% to 26% by weight sericin. Retaining the sericin coating protects the frail fibroin filaments from fraying during textile manufacture. Hence textile grade silk is generally made of sericin coated silk fibroin fibers. Medical grade silkworm silk is used as either as virgin silk suture, where the sericin has not been removed, or as a silk suture from which the sericin has been removed and replaced with a wax or silicone coating to provide a barrier between the silk fibroin and the body tissue and cells.

Hyaluronic acid (HA) (synonymously hyaluron or hyaluronate) is a naturally occurring glucosaminoglycan that has been used as a constituent of a dermal filler for wrinkle reduction and tissue volumizing. Hyaluronan is an anionic, nonsulfated glycosaminoglycan distributed widely throughout connective, epithelial, and neural tissues. Polymeric hyaluronic acid can have a molecular weight of several million Daltons. An individual can typically have about 15 grams of hyaluronan in his body about a third of which every day is degraded by endogenous enzymes and free radicals within a few hours or days and replaced by hyaluronic acid newly synthesized by the body.

Bioconjugate Chemistry, 2010, 21, 240-247: Joem Y., et al., Effect of cross-linking reagents for hyaluronic acid hydrogel dermal fillers on tissue augmentation and regeneration, discusses use of a particular cross-linker HMDA to prepare a cross-linked hyaluronic acid dermal filler, and also discloses use of a variety of hyaluronic acid cross linkers and hyaluronic activators including BDDE and EDC.

Carbohydrate Polymers, 2007, 70, 251-257: Jeon, O., et al., Mechanical properties and degradation behaviors of hyaluronic acid hydrogels cross-linked at various cross-linking densities, discusses properties of hyaluronic acid cross linked with a polyethylene glycol diamine (a PEG-diamine).

J. Am. Chem. Soc., 1955, 77 (14), 3908-3913: Schroeder W., et al., The amino acid composition of Bombyx mori silk fibroin and of Tussah silk fibroin, compares the amino acid compositions of the silk from two silkworm species.

US Patent Application Publication. Pub. No. US 2008/0004421 A1: Chenault, H., et al., Tissue adhesives with modified elasticity discloses an adhesive hydrogel useful as a medical tissue adhesive for example to assist wound closure can be made by preparing a chain extended, multi-arm polyether amine (such as an 8 arm PEG amine) cross linked (using for example PEG 4000 dimesylate) to an oxidized polysaccharide (such as dextran), by mixing the cross linked molecule in a syringe at the point of injection or administration with a hydrogel such as a solution of dextran dialdehyde.

US Patent Application Publication. Pub. No. US 2010/0016886 A1: Lu, H., High swell, long lived hydrogel sealant; discusses reacting a multi-arm amine (i.e. an 9 arm polyethelene glycol (PEG) with an oxidized (i.e. to introduce aldehyde groups) polysaccharide (such as hyaluronic acid), useful for tissue augmentation or a tissue adhesive/sealant.

U.S. Pat. No. 6,903,199 to Moon. T., et al., Crosslinked amide derivatives of hyaluronic acid and manufacturing method thereof discusses cross linking hyaluronic acid with a chitosan or with a deacetylated hyaluronic acid with reactive amide groups, using (for example) EDC or NHS.

International Patent Application WO/2010/123945, Altman, G., et al., Silk fibroin hydrogels and uses thereof discusses silk hydrogels made by, for example, digesting degummed silk hydrogels made by, for example, digesting degummed Bombyx mori silk at 60° C. for 4 hours in 9.3M lithium bromide to thereby obtain a 20% silk solution, an 8% silk solution of which was induced to gel using 23RGD and/or ethanol, which can be present in a hyaluronic acid carrier. Altman also discusses possible use as a dermal filler and to promote wound closure, and (in paragraph [0210]) a silk hydrogel coating on a silk mesh. Altman also discusses silk cross linked to hyaluronic acid (see paragraphs [213] to [220], using various cross linkers.

International Patent Application. Pub. No. WO/2008/008857: Prestwich, G., et al., Tholated macromolecules and methods for making and using thereof discloses a thioethyl ether substituted hyaluronic acid made by oxidating coupling useful, for example, in arthritis treatment.

International Patent Application. Pub. No. WO/2008/008859: Prestwich, G., et al., Macromolecules modified with electrophilic groups and methods of making and using thereof discloses a haloacetate derivative hyaluronic acid reacted with thiol modified hyaluronic acid to make a hydrogel, with various medical uses.

Biomacromolecules, 2010, 11 (9), 2230-2237: Serban, M., et. Al., Modular elastic patches: mechanical and biological effects discusses how to make an elastic patch by cross linking elastin, hyaluronic acid and silk, by adding an aminated hyaluronic acid (made using EDC) with a 20% silk solution and elastin, in PBS with BS3 (bissulfosuccinimidyl suberate, as cross linker) at 37° C. for 12 hours.

Biomaterials, 2008, 29(10), 1388-1399: Serban, M., et al., Synthesis, characterization and chondroprotective properties of a hyaluronan thioethyl ether derivative discusses a viscous 2-thioethyl ether hyaluronic acid derivative solution useful for viscosupplementation in arthritis treatment. The abstract mentions use of hyaluronic acid with multiple thio groups for adhesion prevention.

Methods, 2008, 45, 93-98: Serban, M., et al., Modular extracellular matrices: solutions to the puzzle discusses cross linked thio modified hyaluronic acid hydrogel useful as a semi synthetic extracellular matrix for cell culture.

Biomacromolecules, 2007, 8(9), 2821-2828: Serban, M., et al., Synthesis of hyaluronan haloacetates and biology of novel cross linker free synthetic extracellular matrix hydrogels discusses cross linking haloacetate substituted hyaluronic acids reacted with a thiol substituted hyaluronic acid to make a hydrogel useful for cell culture or adhesion prevention or medical device coating.

Journal of Materials Chemistry, 2009, 19, 6443-6450: Murphy A., et al., Biomedical applications of chemically modified silk fibroin is a review of methods to make silk conjugates, including silk conjugated to oligosaccharides, modified silk and medical uses.

Biomacromolecules, 2004, 5, 751-757: Sohn, S., et al., Phase behavior and hydration of silk fibroin discusses a study of Bombyx mori silk in vitro using osmotic stress, determining that silk I (α-silk) but not silk II (β-sheet, spun silk fiber) is hydrated.

U.S. Pat. No. 8,071,722 to Kaplan, D., et al., Silk Biomaterials and methods of use thereof discloses silk films, use of 9-12 m LiBr to dissolve extracted silk, adding hyaluronic acid to a silk solution to make fibers from the composition. See also eg the Kaplan patents and U.S. Pat. Nos. 7,674,882; 8,178,656; 2010 055438, and; 2011 223153.

US patent application 2011 071239 by Kaplan, D., et al., PH induced silk gels and uses thereof discloses methods for making silk fibroin gel from silk fibroin solution, useful to coat a medical device (see paragraph [0012]), as an injectable gel to fill a tissue void, making an adhesive silk gel (with or without a hyaluronic acid), adhering the adhesive silk gel to a subject for example for use as a wound bioadhesive, a multi-layered silk gel.

US patent application 2009 0202614 by Kaplan, D., et al., Methods for stepwise deposition of silk fibroin coatings discusses layered silk coatings, silk films made using silk fibroin solutions which can include a hyaluronic acid, useful, for example, as wound healing patches, to coat an implantable medical device.

U.S. Pat. No. 4,818,291 to Iwatsuki M., et al., Silk-fibroin and human-fibrinogen adhesive composition discusses surgical adhesive useful in tissue repair made as a mixture of LiBr dissolved silk and fibrinogen.

Implantable, knitted silk fabrics for surgical use are known. See eg US patent applications 2004/0224406 and 2012/0029537. Post operative adhesions are a common occurrence after surgery and are undesirable. For example postoperative intra-abdominal and pelvic adhesions are the leading cause of infertility, chronic pelvic pain, and intestinal obstruction. Adhesions form as a result of the body's natural healing response and imply migration of fibroblasts to the trauma/wound site, cell proliferation, de novo extracellular matrix secretion and wound closing through adhesion formations. Post-operative adhesions can occur at the tissue-tissue interface (i.e. peritendinous tissue adhesion involves adhesion between the repaired tendon and the surrounding tissue) or at a tissue-biomaterial interface, in cases where a biomaterial (i.e. a supporting scaffold) is used to reinforce the mechanical properties of the repaired tissue. For example in hernia repair where a biomaterial mesh is used to reinforce the reconstructed abdominal wall, adhesions commonly form between the mesh and underlying bowel tissue.

Thus there is a need for an implantable biomaterial mesh that can decrease or eliminate formation of post-operative adhesions.

### SUMMARY

The present invention meets these needs and provides silk based medical devices that can reduce or prevent post-operative tissue to tissue or tissue to scaffold adhesion formation. Important to the invention was discovery of a biocompatible material that does not promote cell attachment, provides a smooth surface that hinders cell attachment, eliminates the introduction of foreign chemical agents, exploit silk's intrinsic physical cross linking capacity via hydrogen-bond mediated beta-sheet formation; and provides a robust, pliable, and user friendly implantable medical device.

The present invention also includes an entirely silk based self adherent medical devices. This device is: biocompatible and can stick (adhere) to a physiological surface (such as skin or other tissue surface); provides a smooth surface that can prevent cell adherence and/or tissue abrasions; circumvent the introduction of any external agents or chemicals; makes use of silk's intrinsic physical crosslinking capacity via hydrogen-bond mediated beta-sheet formation; and (e) robust, pliable, cost-efficient and a user friendly medical device.

An embodiment of the present invention is a laminate, implantable silk medical device having a first layer comprising a knitted silk fabric, the first later having a top side and a bottom side, and a second layer comprising a silk film or sponge fused to at least a portion of the bottom side of the first layer, thereby obtaining a laminate, implantable silk medical device. The silk film or sponge can comprise silk and a compound selected from the group consisting of polyethylene glycol, ethylene oxide, propylene oxide block copolymer, hyaluronic acid, dextran, and alginate and salts and combinations thereof. Additionally, the silk film or sponge can be water resistant and the silk film can be fused to the silk fabric by drying the silk film or sponge after placing the silk film or sponge onto the silk fabric.

Additional embodiments of the present invention can include an implantable silk medical device with or without pores, knitted using one to 36 filament silk yarn prepared at a various twist rates; an implantable silk medical device which is about 0.5 mm to about 4 mm thick; an implantable silk medical device knitted as a flat sheet with a top side and a bottom side wherein the bottom is has a low profile, anti-adhesive surface, and; a laminate, implantable silk medical device comprising: (a) a first layer comprising a knitted silk fabric, the first later having a top side and a bottom side; (b) a second joining layer comprising a knitted, non-silk fabric having a top side and a bottom side, the second joining layer joining the bottom side of the first layer to the top side of the second joining layer and the bottom side of the second joining layer a top side of a third sacrificial layer, and; (c) the third sacrificial layer comprising a knitted, non-silk fabric having a top side and a bottom side, the top side of the third sacrificial layer attached to at least a portion of the bottom side of the second layer, thereby obtaining a laminate, implantable silk medical device, wherein the first layer biodegrades over about 1 years to about 3 years after implantation of the device, and the second joining layer biodegrades over about 10 to 30 days after implantation of the device biodegradation of the second joining layer thereby releasing the third sacrificial layer from indirect attachment to the first layer through the second joining layer.

Another embodiment of the present invention is a process for making a laminate, implantable silk medical device by (a) knitting a fabric from sericin depleted silk thereby making a first layer having a top side and a bottom side, (b) preparing a silk solution by dissolving silk into a solvent; (c) casting a silk film or sponge from the silk solution; (d) treating the silk film or sponge so that at least one side of the silk film is water resistant, thereby forming a second layer; and (e) fusing the second layer to at least a portion of the bottom side of the first layer, thereby obtaining a laminate, implantable silk medical device.

The present invention also includes a method for providing tissue support and reducing adhesion formation by implanting the device, including an abdominal surgical method comprising the step of implanting the device.

A detailed embodiment of the present invention can be a laminate, implantable silk medical device comprising: (a) a first layer comprising a water resistant, non-adherent silk film, the first layer having a top side and a bottom side, and; (b) a second layer comprising a water soluble, adherent silk film or sponge formed on or placed on the top side of the first layer, thereby obtaining a laminate, implantable silk medical device.

Additional embodiments of the present invention can be: an implantable silk medical device with an average pore size of about 4 mm by about 4 mm, knitted using six or nine filament silk yard prepared at a twist rate of 2(6S) 3(3(Z); an implantable silk medical device which is about 3 mm to about 4 mm thick made with a pick density of about 26 picks per centimeter; an implantable silk medical device knitted as a flat sheet with a top side and a bottom side wherein the bottom is has a smooth, anti-adhesive surface made with a pick density of about 18 picks per centimeter, and; a laminate, implantable silk medical device comprising: (a) a first layer comprising a knitted silk fabric, the first later having a top side and a bottom side; (b) a second joining layer comprising a knitted, non-silk fabric having a top side and a bottom side, the second joining layer joining the bottom side of the first layer to the top side of the second joining layer and the bottom side of the second joining layer a top side of a third sacrificial layer, and; (c) the third sacrificial layer comprising a knitted, non-silk fabric having a top side and a bottom side, the top side of the third sacrificial layer attached to at least a portion of the bottom side of the second layer, thereby obtaining a laminate, implantable silk medical device, wherein the first layer biodegrades over about 1 years to about 3 years after implantation of the device, and the second joining layer biodegrades over about 30 days after implantation of the device biodegradation of the second joining layer thereby releasing the third sacrificial layer from indirect attachment to the first layer through the second joining layer.

The present invention also includes a laminate, implantable silk medical device comprising: (a) a first base layer comprising a knitted silk fabric, the first layer having a top side and a bottom side; (b) a second anti-adhesive layer comprising a knitted silk fabric having a top side and a bottom side, the second anti-adhesive layer being attached at least in part on the bottom side of the first layer, wherein the first and second layer biodegrades over about 1 years to about 3 years after implantation of the device. The present invention also includes a laminate, implantable silk medical device comprising: (a) a first base layer comprising a knitted silk fabric, the first layer having a top side and a bottom side, and; (b) a second sacrificial layer comprising a knitted, non-silk fabric having a top side and a bottom side, the second sacrificial layer being attached at least in part on the bottom side of the first layer, wherein the first layer biodegrades over about 1 years to about 3 years after implantation of the device, and the second sacrificial layer biodegrades over about 10 to 30 days after implantation of the device. The present invention also includes a laminate, implantable silk medical device comprising: (a) a first base layer comprising a knitted silk fabric, the first layer having a top side and a bottom side; (b) a second (middle) layer comprising a knitted, non-silk fabric having a top side and a bottom side, the second joining layer joining the bottom side of the first layer to the top side of the second joining layer and the bottom side of the second joining layer a top side of a third sacrificial layer, and; (c) the third detaching layer comprising a knitted, non-silk or silk fabric having a top side and a bottom side, the top side of the third sacrificial layer attached to at least a portion of the bottom side of the second layer, thereby obtaining a laminate, implantable silk medical device, wherein the first layer biodegrades over about 1 years to about 3 years after implantation of the device, the second joining layer biodegrades over about 10 to 30 days after implantation, releasing the third sacrificial layer from tissue attachment, the thirds sacrificial layer biodegrading over about 10 days to about 3 years after implantation of the device.

The present invention also includes a laminate, implantable silk medical device comprising: (a) a first base layer comprising a knitted silk fabric, the first later having a top side and a bottom side, and; (b) a second layer comprising a silk film or sponge fused to at least a portion of the bottom side of the first layer, thereby obtaining a laminate, implantable silk medical device, wherein the silk film is water resistant, wherein the silk film or sponge is fused to the silk fabric by drying the silk film or sponge after placing the silk film onto the silk fabric.

The present invention also includes a process for making a laminate, implantable silk medical device, the process comprising (a) knitting a fabric from sericin depleted silk thereby making a first layer having a top side and a bottom side, and; (b) preparing a silk solution by dissolving silk into a solvent; (c) casting a silk film or sponge from the silk solution; (d) treating the silk film or sponge so that at least one side of the silk film is water resistant, thereby forming a second layer; and (e) fusing the second layer to at least a portion of the bottom side of the first layer, thereby obtaining a laminate, implantable silk medical device.

The present invention also includes a laminate, implantable silk medical device comprising: (a) a first layer comprising a water resistant, non-adherent silk film or sponge, the first layer having a top side and a bottom side, and; (b) a second layer comprising a water soluble, adherent silk film or sponge formed on or placed on the top side of the first layer, thereby obtaining a laminate, implantable silk medical device, wherein the silk film or sponge comprises silk and a compound selected from the group consisting of polyethylene glycol, ethylene oxide, propylene oxide block copolymer, hyaluronic acid, dextran, and alginate and salts and combinations thereof.

### DRAWINGS

Aspects of the present invention are illustrated by the following drawings.

FIG. 1 illustrates the procedure for casting a silk form from a silk solution to thereby make a water resistant silk film. The middle drawing in FIG. 1 shows the silk solution being dispensed from a pipette. “EtOH” in FIG. 1 means application of ethanol to the silk film.

FIG. 2 illustrates the procedure for making a multi laminate medical device using the water resistant silk film made by the FIG. 1 process. In FIG. 2 the water resistant silk film is shown fused onto a knitted silk mesh (the particular knitted silk mesh used was SERI® Surgical Scaffold, available from Allergan, Irvine, Calif.).

FIG. 3 is a graph obtained by use of FTIR showing on the x axis the absorbance wavelength (nm) and on the y axis the absorbance (arbitrary units or AU) confirming beta sheet induction through silk film treatment with the ethanol solution.

FIG. 4 shows on the left hand side of FIG. 4 a side view photograph and on the right hand side of FIG. 4 a top view photograph of the water resistant silk film made by the process of FIG. 1.

FIG. 5 is a pictorial representation of how the silk film made by the process of FIG. 1 can be used to wrapped around a portion of a tendon so as to isolate the tendon from adjacent tissues.

FIG. 6 top—shows on the left hand side of FIG. 6 a bottom view photograph (the “smooth side”) of a multi laminate medical device comprising a water resistant silk film fused to the knitted silk fabric. The right hand side of FIG. 6 is a top view photograph (the “rough side”) of the multi laminate silk device. FIG. 6 bottom—contrasts the device comprising of a water resistant silk film fused to the knitted silk fabric with the device comprising of a water resistant silk sponge fused to the knitted silk fabric.

FIG. 7 is a pictorial representation showing in the top portion of FIG. 7 knit characteristics of the knitted silk fabric used (SERI® Surgical Scaffold), and in the bottom portion of FIG. 7.

FIG. 8 is a pictorial representation of the use of the fused silk-film mesh medical device for post-operative adhesion prevention in an abdominal wall repair model.

FIG. 9 is an illustration of the casting process of a double layered self-adherent silk film.

FIG. 10 is a graph obtained by use of FTIR showing on the x axis the absorbance wavelength (nm) and on the y axis the absorbance (AU) confirming beta sheet induction through silk film treatment with the ethanol solution.

FIG. 11 presents two photographs of a multi laminate (two layers of silk film) medical device, showing in the left hand side photograph adherence to the top of a Petri dish and in the right hand side photograph adherence to a moistened nitrile surgical glove.

FIG. 12 is a pictorial illustration of the silk film adherence mechanism to wet or moist surfaces. The hydrophilicity of the contact surface probably triggers silk fibroin structural rearrangements that lead to the reorientation of the hydrophilic and hydrophobic regions of the protein to promote the most energetically favorable interactions.

FIG. 13 presents two bar graphs: evaluation of cell numbers after 48 hours (the upper graph in FIG. 13) and after 6 days (the lower graph in FIG. 13) incubation on different biomaterial formulations, by colorimetric MTS (3-(4,5-dimethylthiazol-2-yl)-5-(3-carboxymethoxyphenyl)-2-(4-sulfophenyl)-2H-tetrazolium) (tetrazolium dye) assay.

FIG. 14 comprises five bar graphs showing the additive dose dependent cell responses on different biomaterial (second layer) formulations (24 hour incubation with 2×10⁵ cells/well). The dotted box in each of the five FIG. 14 graphs shows the preferred second layer for the formulation set forth by that bar graph.

FIG. 15 is a diagram showing the knit pattern used to make the single bed 102 (base layer) device.

FIG. 16 is showing the appearance of the technical back and technical front of the single bad 102 (base layer) device that was knitted with the pattern shown in FIG. 15.

FIG. 17 is a diagram showing the knit pattern used to make satin devices—both anti-adhesive and sacrificial (prototypes SS-P02-02-01, SS-P02-02-02, SS-P02-02-04 and SS-P02-02-10).

FIG. 18 is showing the appearance of the technical back and technical front of a representative anti-adhesive satin device (SS-P02-02-01) that was knitted with the pattern shown in FIG. 17.

FIG. 19 is a diagram showing the knit pattern used to make “shag carpet” devices.

FIG. 20 is showing the appearance of the technical back, technical front and cross-section of a representative “shag carpet” device (SS-P02-03-09) that was knitted with the pattern shown in FIG. 19.

FIG. 21 is showing the appearance of the technical back and technical front of a representative sacrificial layer satin device (SS-P02-02-10) that was knitted with the pattern shown in FIG. 17.

FIG. 22 is a depiction of the detachable layer device concept.

FIG. 23 is a diagram showing the knit pattern used to make representative detachable layer devices (SS-P04-01 and SS-P04-02-0X).

FIG. 24 is showing the appearance of the technical back, technical front and cross-section of a representative detachable layer device (SS-P04-03).

FIG. 25 is a bar graph showing the measured thickness in millimeters of the SERI® Surgical Scaffold (“SERI® Standard”) and six of the devices made.

FIG. 26 is a bar graph showing the measured burst strength (in MPa) of the SERI® Surgical Scaffold (“SERI® Standard”) and the same six devices measured in FIG. 25.

FIG. 27 is a bar graph showing the stiffness (in Newtons per millimeter) of the SERI® Surgical Scaffold (“SERI® Standard”) and the same six devices measured in FIG. 25.

FIG. 28 is a bar graph showing the measured suture pull out strength (in Newtons per suture) of the SERI® Surgical Scaffold (“SERI® Standard”) and the same six devices measured in FIG. 25.

FIG. 29 is a bar graph showing the maximum load in the machine (fabric length) direction measured in Newtons for the SERI® Surgical Scaffold (“SERI® Standard”) and for the same six devices measured in FIG. 25.

FIG. 30 is a bar graph showing the measured percent elongation at break in the machine (fabric length) direction for the SERI® Surgical Scaffold (“SERI® Standard”) and for the same six devices measured in FIG. 25.

FIG. 31 is a bar graph showing shows the maximum load in the course (fabric width) direction in Newtons for the SERI® Surgical Scaffold (“SERI® Standard”) and for the same six devices measured in FIG. 25.

FIG. 32 is a bar graph shows the percent elongation at break in the course (fabric width) direction for the SERI® Surgical Scaffold (“SERI® Standard”) and for the same six devices measured in FIG. 25.

### DESCRIPTION

The present invention is based on the discovery of laminate silk medical devices that can be implanted to separate adjoining tissues, provide soft tissue support and/or reduce formation of adhesions.

The silk films and the silk fabrics set forth herein can be made from silkworm cocoons substantially depleted of sericin. A preferred source of raw silk is from the silkworm B. mori. Other sources of silk include other strains of Bombycidae including Antheraea pemyi, Antheraea yamamai, Antheraea mylitta, Antheraea assama, and Philosamia cynthia ricini, as well as silk producing members of the families Satumidae, Thaumetopoeidae, and silk-producing members of the order Araneae. Suitable silk can also be obtained from other spider, caterpillar, or recombinant sources. Methods for performing sericin extraction have been described in pending U.S. patent application Ser. No. 10/008,924, U.S. Publication No. 2003/0100108, Matrix for the production of tissue engineered ligaments, tendons and other tissue.

Extractants such as urea solution, hot water, enzyme solutions including papain among others which are known in the art to remove sericin from fibroin would also be acceptable for generation of the silk. Mechanical methods may also be used for the removal of sericin from silk fibroin. This includes but is not limited to ultrasound, abrasive scrubbing and fluid flow. The rinse post-extraction is conducted preferably with vigorous agitation to remove substantially any ionic contaminants, soluble, and insoluble debris present on the silk as monitored through microscopy and solution electrochemical measurements. A criterion is that the extractant predictably and repeatably remove the sericin coat of the source silk without significantly compromising the molecular structure of the fibroin. For example, an extraction may be evaluated for sericin removal via mass loss, amino acid content analysis, and scanning electron microscopy. Fibroin degradation may in turn be monitored by FTIR analysis, standard protein gel electrophoresis and scanning electron microscopy.

In certain cases, the silk utilized for making the composition has been substantially depleted of its native sericin content (i.e. ≦4% (w/w) residual sericin in the final extracted silk). Alternatively, higher concentrations of residual sericin may be left on the silk following extraction or the extraction step may be omitted. In preferred aspects of this embodiment, the sericin-depleted silk fibroin has, e.g. about 0% to about 4% (w/w) residual sericin. In the most preferred aspects of this embodiment, the sericin-depleted silk fibroin has, e.g. about 1% to 3% (w/w) residual sericin.

In certain cases, the silk utilized for generation of a medical device within the scope of the present invention is entirely free of its native sericin content. As used herein, the term “entirely free (i.e. “consisting of” terminology) means that within the detection range of the instrument or process being used, the substance cannot be detected or its presence cannot be confirmed.

The water soluble or dissolved silk can be prepared by a 4 hour solubilization (process of silk into solution) at 60° C. of pure silk fibroin at a concentration of 200 g/L in a 9.3 M aqueous solution of lithium bromide to a silk concentration of 20% (w/v). This process may be conducted by other means provided that they deliver a similar degree of dissociation to that provided by a 4 hour solubilization at 60° C. of pure silk fibroin at a concentration of 200 g/L in a 9.3 M aqueous solution of lithium bromide. The primary goal of this is to create uniformly and repeatably dissociated silk fibroin molecules to ensure similar fibroin solution properties and, subsequently, device properties. Less substantially dissociated silk solution may have altered gelation kinetics resulting in differing final gel properties. The degree of dissociation may be indicated by Fourier-transform Infrared Spectroscopy (FTIR) or x-ray diffraction (XRD) and other modalities that quantitatively and qualitatively measure protein structure. Additionally, one may confirm that heavy and light chain domains of the silk fibroin dimer have remained intact following silk processing and dissolution. This may be achieved by methods such as standard protein sodium-dodecyl-sulfate polyacrylamide gel electrophoresis (SDS-PAGE) which assess molecular weight of the independent silk fibroin domains.

System parameters which may be modified in the initial dissolution of silk include but are not limited to solvent type, silk concentration, temperature, pressure, and addition of mechanical disruptive forces. Solvent types other than aqueous lithium bromide may include but are not limited to aqueous solutions, alcohol solutions, 1,1,1,3,3,3-hexafluoro-2-propanol, and hexafluoroacetone, 1-butyl-3-methylimidazolium. These solvents may be further enhanced by addition of urea or ionic species including lithium bromide, calcium chloride, lithium thiocyanate, zinc chloride, magnesium salts, sodium thiocyanate, and other lithium and calcium halides would be useful for such an application. These solvents may also be modified through adjustment of pH either by addition of acidic of basic compounds.

The medical devices disclosed herein are preferably biodegradable, bioerodible, and/or bioresorbable. In a particular embodiment the medical device (for example as a silk film) can entirely or substantially biodegrade between about 10 days to about 120 days after implantation. In another embodiment the medical device (for example formed as a laminate silk device comprising both a silk film and a knitted silk fabric) can entirely or substantially biodegrade over a period of time between about 3 years or about 4 years after implantation.

Aspects of the present specification provide, in part, a silk film having a transparency and/or translucency. Transparency (also called pellucidity or diaphaneity) is the physical property of allowing light to pass through a material, whereas translucency (also called translucence or translucidity) only allows light to pass through diffusely. The opposite property is opacity. Transparent materials are clear, while translucent ones cannot be seen through clearly. The silk films disclosed herein may, or may not, exhibit optical properties such as transparency and translucency. In certain cases, e.g., superficial line filling, it would be an advantage to have an opaque silk film. In other cases such as development of a lens or a “humor” for filling the eye, it would be an advantage to have a translucent silk film. These properties could be modified by affecting the structural distribution of the silk film. Factors used to control a hydrogel's optical properties include, without limitation, silk fibroin concentration, gel crystallinity, and silk homogeneity.

When light encounters a material, it can interact with it in several different ways. These interactions depend on the nature of the light (its wavelength, frequency, energy, etc.) and the nature of the material. Light waves interact with an object by some combination of reflection, and transmittance with refraction. As such, an optically transparent material allows much of the light that falls on it to be transmitted, with little light being reflected. Materials which do not allow the transmission of light are called optically opaque or simply opaque.

In an embodiment, a silk film is optically transparent. In aspects of this embodiment, a silk film transmits, e.g., between about 75% to about 100% of the light. In some preferred aspects of this embodiment, a silk film transmits, e.g., between about 80% to about 90% of the light. In the most preferred aspects of this embodiment, a silk film transmits, e.g., between about 85% to about 90% of the light.

In an embodiment, a silk sponge is optically transparent. In aspects of this embodiment, a silk sponge transmits, e.g., between about 75% to about 100% of the light. In some preferred aspects of this embodiment, a silk film transmits, e.g., between about 80% to about 90% of the light. In the most preferred aspects of this embodiment, a silk sponge transmits, e.g., between about 85% to about 90% of the light.

Aspects of the present specification provide, in part, a medical device comprising a hyaluronan. As used herein, the term “hyaluronic acid” is synonymous with “HA”, “hyaluronic acid”, and “hyaluronate” refers to an anionic, non-sulfated glycosaminoglycan polymer comprising disaccharide units, which themselves include D-glucuronic acid and D-N-acetylglucosamine monomers, linked together via alternating β-1,4 and β-1,3 glycosidic bonds and pharmaceutically acceptable salts thereof. Hyaluronan can be purified from animal and non-animal sources. Polymers of hyaluronan can range in size from about 5,000 Da to about 20,000,000 Da. Any hyaluronan is useful in the compositions disclosed herein with the proviso that the hyaluronan improves a condition of the skin, such as, e.g., hydration or elasticity. Non-limiting examples of pharmaceutically acceptable salts of hyaluronan include sodium hyaluronan, potassium hyaluronan, magnesium hyaluronan, calcium hyaluronan, and combinations thereof.

Aspects of the present specification provide, in part, a composition comprising a crosslinked matrix polymer. As used herein, the term “crosslinked” refers to the intermolecular physical or chemical bonds joining the individual polymer molecules, or monomer chains, into a more stable structure like a gel. As such, a crosslinked matrix polymer has at least one intermolecular physical or chemical bond joining at least one individual polymer molecule to another one. Matrix polymers disclosed herein may be chemically crosslinked using dialdehydes and disufides crosslinking agents including, without limitation, multifunctional PEG-based cross linking agents, divinyl sulfones, diglycidyl ethers, and bis-epoxides. Non-limiting examples of hyaluronan crosslinking agents include divinyl sulfone (DVS), 1,4-butanediol diglycidyl ether (BDDE), 1,2-bis(2,3-epoxypropoxy)ethylene (EGDGE), 1,2,7,8-diepoxyoctane (DEO), biscarbodiimide (BCDI), pentaerythritol tetraglycidyl ether (PETGE), adipic dihydrazide (ADH), bis(sulfosuccinimidyl)suberate (BS), hexamethylenediamine (HMDA), 1-(2,3-epoxypropyl)-2,3-epoxycyclohexane, or combinations thereof.

Aspects of the present specification provide, in part, a composition comprising a crosslinked matrix polymer having a degree of crosslinking. As used herein, the term “degree of crosslinking” refers to the percentage of matrix polymer monomeric units that are bound to a cross-linking agent, such as, e.g., the disaccharide monomer units of hyaluronan. Thus, a composition that that has a crosslinked matrix polymer with a 4% degree of crosslinking means that on average there are four crosslinking molecules for every 100 monomeric units. Every other parameter being equal, the greater the degree of crosslinking, the harder the gel becomes. Non-limiting examples of a degree of crosslinking include about 1% to about 15%.

In an embodiment, a composition comprises an uncrosslinked hyaluronan where the uncrosslinked hyaluronan comprises a combination of both high molecular weight hyaluronan and low molecular weight hyaluronan in a ratio of about 20:1, about 15:1, about 10:1, about 5:1, about 1:1, about 1:5 about 1:10, about 1:15, or about 1:20.

In another embodiment, a composition comprises an uncrosslinked hyaluronan where the uncrosslinked hyaluronan comprises a combination of both high molecular weight hyaluronan and low molecular weight hyaluronan, in various ratios. As used herein, the term “high molecular weight hyaluronan” refers to a hyaluronan polymer that has a molecular weight of 1,000,000 Da or greater. Non-limiting examples of a high molecular weight hyaluronan include a hyaluronan of about 1,500,000 Da, a hyaluronan of about 2,000,000 Da, a hyaluronan of about 2,500,000 Da, a hyaluronan of about 3,000,000 Da, a hyaluronan of about 3,500,000 Da, a hyaluronan of about 4,000,000 Da, a hyaluronan of about 4,500,000 Da, and a hyaluronan of about 5,000,000 Da. As used herein, the term “low molecular weight hyaluronan” refers to a hyaluronan polymer that has a molecular weight of less than 1,000,000 Da. Non-limiting examples of a low molecular weight hyaluronan include a hyaluronan of about 200,000 Da, a hyaluronan of about 300,000 Da, a hyaluronan of about 400,000 Da, a hyaluronan of about 500,000 Da, a hyaluronan of about 600,000 Da, a hyaluronan of about 700,000 Da, a hyaluronan of about 800,000 Da, and a hyaluronan of about 900,000 Da.

In other aspects of this embodiment, a composition comprises a crosslinked hyaluronan where the crosslinked hyaluronan has a mean molecular weight of, e.g., about 1,000,000 Da, about 1,500,000 Da, about 2,000,000 Da, about 2,500,000 Da, about 3,000,000 Da, about 3,500,000 Da, about 4,000,000 Da, about 4,500,000 Da, or about 5,000,000 Da. In yet other aspects of this embodiment, a composition comprises a crosslinked hyaluronan where the crosslinked hyaluronan has a mean molecular weight of, e.g., at least 1,000,000 Da, at least 1,500,000 Da, at least 2,000,000 Da, at least 2,500,000 Da, at least 3,000,000 Da, at least 3,500,000 Da, at least 4,000,000 Da, at least 4,500,000 Da, or at least 5,000,000 Da. In still other aspects of this embodiment, a composition comprises a crosslinked hyaluronan where the crosslinked hyaluronan has a mean molecular weight of, e.g., about 1,000,000 Da to about 5,000,000 Da, about 1,500,000 Da to about 5,000,000 Da, about 2,000,000 Da to about 5,000,000 Da, about 2,500,000 Da to about 5,000,000 Da, about 2,000,000 Da to about 3,000,000 Da, about 2,500,000 Da to about 3,500,000 Da, or about 2,000,000 Da to about 4,000,000 Da.

In other aspects of this embodiment, a composition comprises an uncrosslinked hyaluronan where the uncrosslinked hyaluronan has a mean molecular weight of, e.g., about 1,000,000 Da, about 1,500,000 Da, about 2,000,000 Da, about 2,500,000 Da, about 3,000,000 Da, about 3,500,000 Da, about 4,000,000 Da, about 4,500,000 Da, or about 5,000,000 Da. In yet other aspects of this embodiment, a composition comprises an uncrosslinked hyaluronan where the uncrosslinked hyaluronan has a mean molecular weight of, e.g., at least 1,000,000 Da, at least 1,500,000 Da, at least 2,000,000 Da, at least 2,500,000 Da, at least 3,000,000 Da, at least 3,500,000 Da, at least 4,000,000 Da, at least 4,500,000 Da, or at least 5,000,000 Da. In still other aspects of this embodiment, a composition comprises an uncrosslinked hyaluronan where the uncrosslinked hyaluronan has a mean molecular weight of, e.g., about 1,000,000 Da to about 5,000,000 Da, about 1,500,000 Da to about 5,000,000 Da, about 2,000,000 Da to about 5,000,000 Da, about 2,500,000 Da to about 5,000,000 Da, about 2,000,000 Da to about 3,000,000 Da, about 2,500,000 Da to about 3,500,000 Da, or about 2,000,000 Da to about 4,000,000 Da. In further aspects, a composition comprises an uncrosslinked hyaluronan where the uncrosslinked hyaluronan has a mean molecular weight of, e.g., greater than 2,000,000 Da and less than about 3,000,000 Da, greater than 2,000,000 Da and less than about 3,500,000 Da, greater than 2,000,000 Da and less than about 4,000,000 Da, greater than 2,000,000 Da and less than about 4,500,000 Da, greater than 2,000,000 Da and less than about 5,000,000 Da.

### EXAMPLES

The following examples illustrate embodiments of the present invention.

### Example 1

### Preparation of a Silk Based Biomaterial Useful as an Adhesion Barrier

The materials used in this Example 1 to make a silk based biomaterial useful as an adhesion barrier included: an aqueous silk fibroin solution (7-12% w/v concentration of silk); sterile 60-mm Petri dishes (used as casting molds); ethanol solution 90% v/v, and; a knitted silk fabric (the particular knitted silk fabric used was SERI Surgical Scaffold. SERI® Surgical Scaffold is available from Allergan, Inc., Irvine, Calif.). SERI® Surgical Scaffold is an embodiment of the knitted silk medical devices set forth in U.S. patent application Ser. Nos. 13/715,872; 13/587,040; 13/843,519; 13/088,706, and; Ser. No. 12/680,404.

As a first step to obtain a solution of water-soluble silk fibroin, either Bombyx Mori silk cocoons or silk fibroin yarn made by processing Bombyx Mori silk cocoons were soaked in a warm basic solution to thereby remove the immunogenic protein sericin naturally present on the silkworm silk. The sericin depleted silk was then digested (solubilized) by dissolving the sericin depleted silk in 9.3M LiBr followed by dialysis into an aqueous solution. The amino acid composition of Bombyx Mori silk fibroin shows a low amount of aspartic acid/glutamic acid (carboxylic groups), even lower amount of lysine (amine groups) and a high amount of serine (hydroxyl groups). Silk beta-sheet formation can be induced with accelerants (pH, temperature, vortexing, sonication, ethanol treatment, etc.).

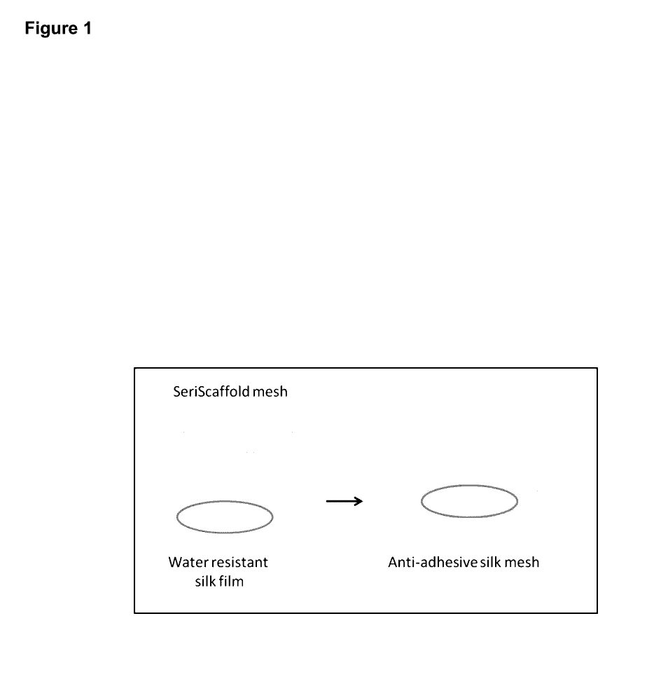

A first device was made as follows. Silk fibroin solution (1 ml) was cast on the bottom of an inverted 60 mm Petri dish and allowed to dry between 2-12 hours (see FIG. 1). The dried films were then immersed for two 2 hours in the ethanol solution to induce beta-sheet formation in the silk.

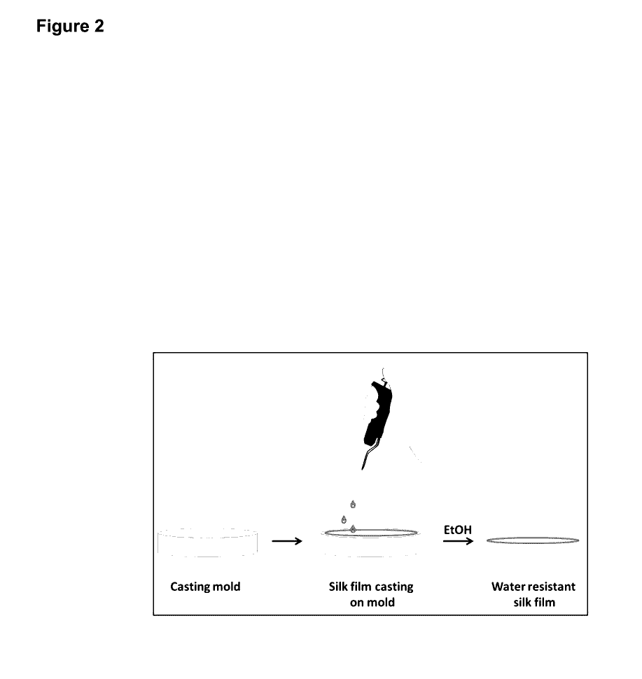

A second device was made as follows. Silk fibroin films cast as described above were allowed to dry for 50 minutes in a laminar flow hood then, prior to complete drying of the surface, were overlayed with precut SERI® Surgical Scaffold meshes (4×5 cm) (see FIG. 2). The film was allowed to fuse with the mesh for 2-12 hours, then the construct was immersed in ethanol solution for 2 hours to induce physical crosslinking via beta sheets.

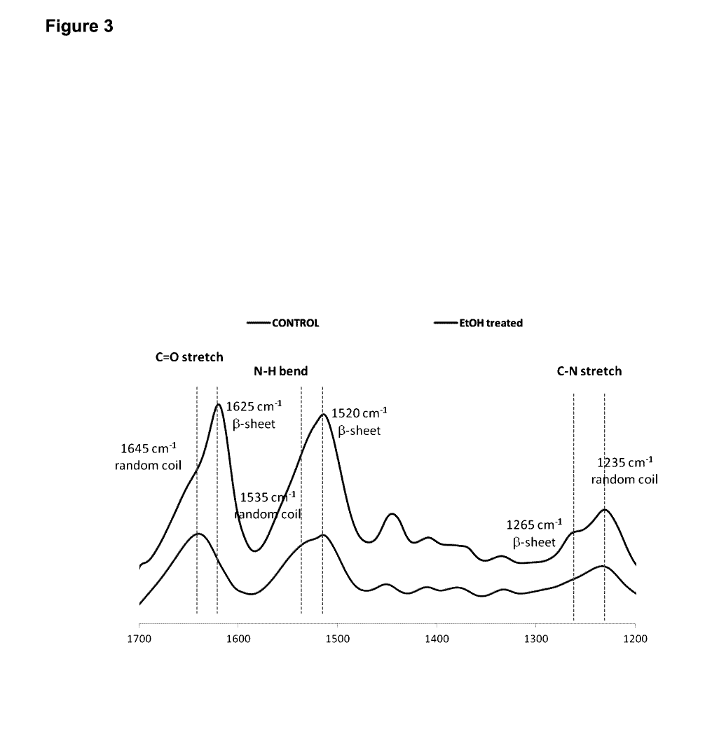

For both devices made in this Example 1, the ability of silk to become water resistant by physical crosslinking of the silk molecules was made use of. Through this cross linking process, the silk fibroin protein underwent structural rearrangements to a beta-sheet rich conformation. Temperature, pH, ionic strength and treatment with polar agents such as alcohols are all factors known to induce such structural transitions. For the two devices made in this Example 1, beta sheet formation was induced via ethanol treatment (see FIG. 3).

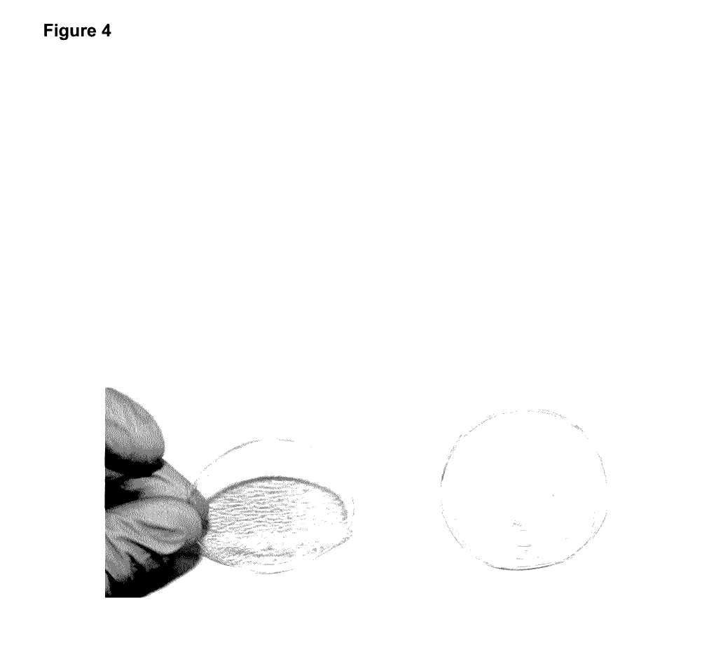

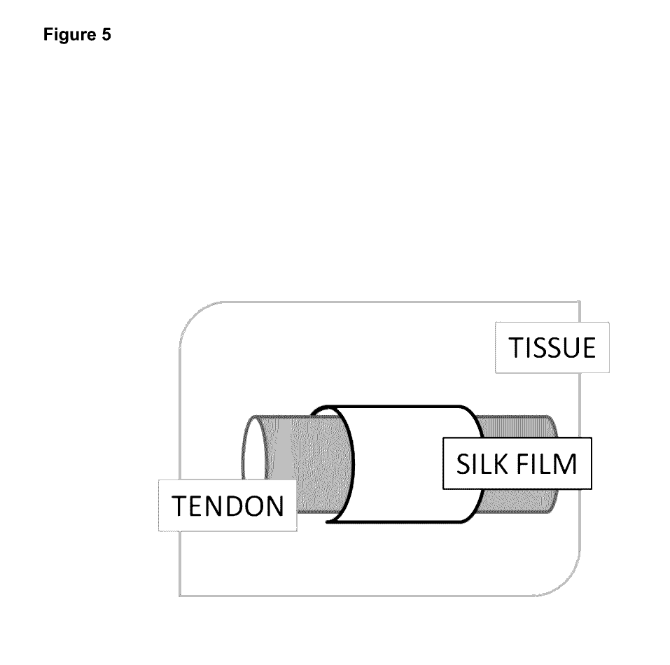

The first device was a monolayer of transparent water-resistant silk film, as shown by FIG. 4. The thickness of the film was controllable and depended on the silk fibroin solution concentration and the casting area. We found that an 8% w/v silk fibroin solution cast on a 4.6 cm diameter mold would yield a 50 μm tick film. The film was pliable, moldable, stretchable, with good mechanical integrity (average maximum load of 8.8±1.9 N for a 50 μm thick film versus an average maximum load of 71.7±1.0 N of SERI® Surgical Scaffold) and can be used to wrap the target tissue (i.e. tendon) to isolate it from the surrounding tissues to with it may non-specifically adhere (FIG. 5). Additionally, the first device can be used in conjunction with other devices (meshes, sheets). Moreover, the transparency of device 1 is a convenient feature as it allows the user to correctly evaluate the positioning of the device 1 film on the tissue.

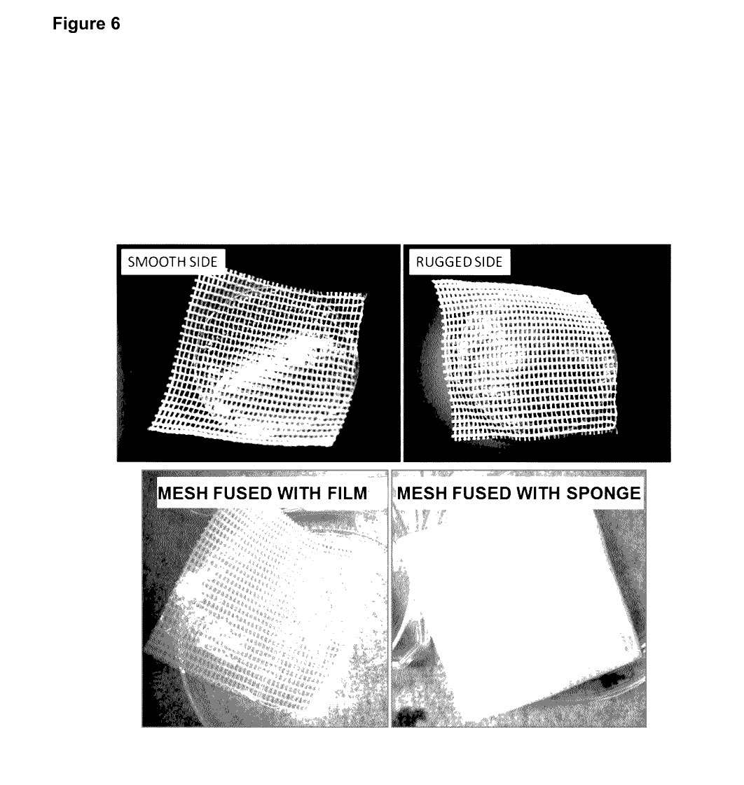

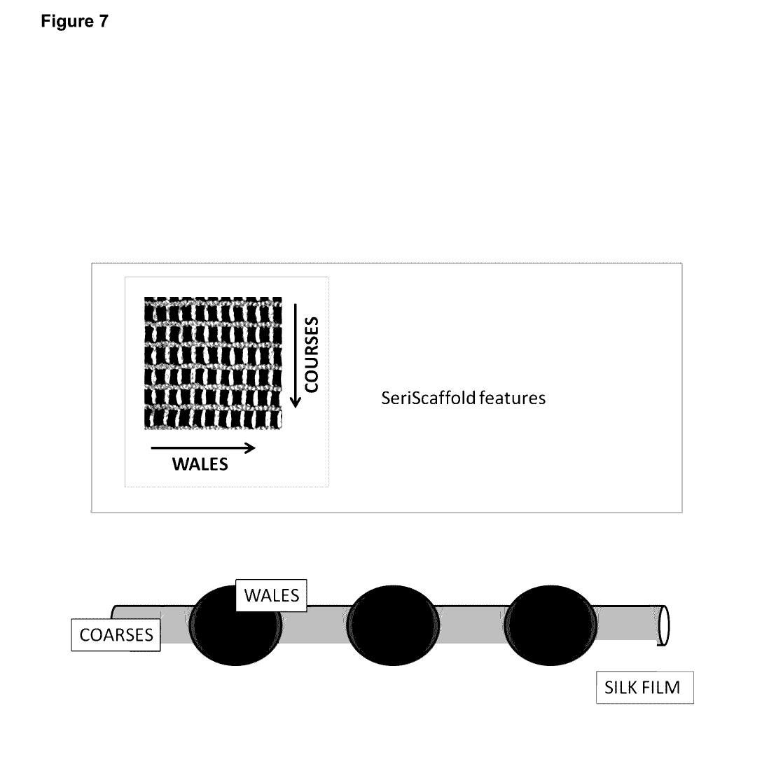

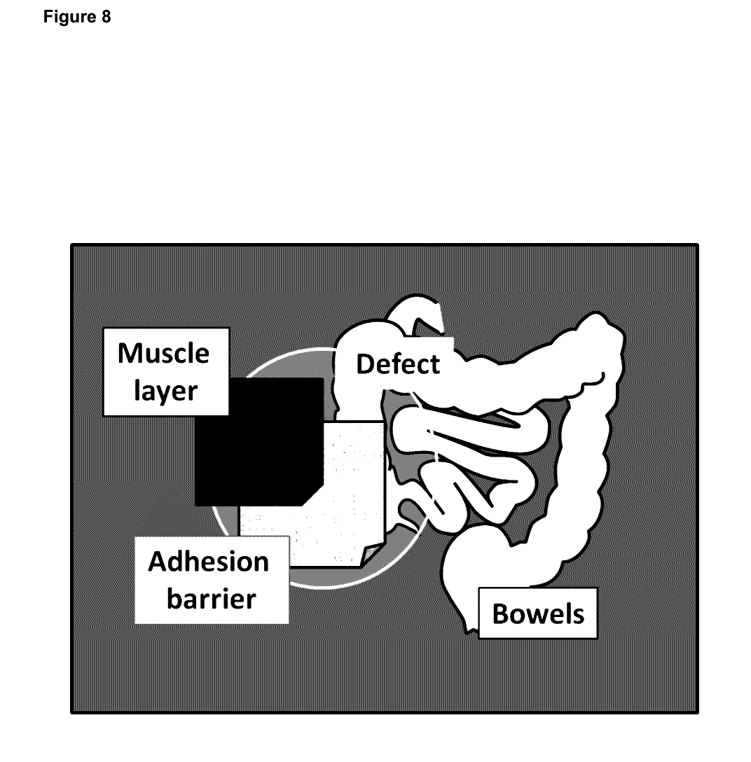

The second device made in this Example 1 consisted of a single layer silk film fused with the SERI® Surgical Scaffold (see FIG. 6). The fusion of the silk with the mesh was driven by the partial encasing of the mesh filaments by the silk solution prior its complete drying (FIG. 7). After complete drying of the film the construct was treated with ethanol solution to render it water insoluble via beta-sheet formation. The key features of this second device were: (a)—its smooth surface on one side and (b)—the rugged surface provided by the mesh pores on the other side. In the case of the abdominal wall repair for example, the smooth side is intended to contact the bowel and prevent adhesion formations, while the rugged surface will face the abdominal wall and will integrate well with the surrounding tissue by promoting cells to adhere to its groove (FIG. 8).

Both device 1 and device 2 have the advantage of being both entirely silk fibroin based. The sterility of both these devices can be ensured either by using autoclaved silk fibroin solution for film casting (and fusing them with sterile meshed for device 2) or via ethylene oxide sterilization. Moreover, both devices are compatible to be used with a variety of other mesh medical such as Vicryl and Mersilene. These devices: (a)—are biocompatible and do not intrinsically sustain cell attachment as previously established by large bodies of scientific literature; (b)—provide a smooth surface that further hinders cell attachment; (c)—do not contain any “foreign” chemical agents; (d)—are physically crosslinked through intra- and inter-molecular beta-sheets; and (e)—are robust, drapable and easy to handle.

### Example 2

### Self-Adherent Silk Based Biomaterials

The materials used in this Example 2 included: an aqueous silk fibroin solution (7-12% w/v) made by the same methods set forth in Example 2; sterile 60-mm Petri dishes (used as casting molds), and; an ethanol solution 90% v/v.

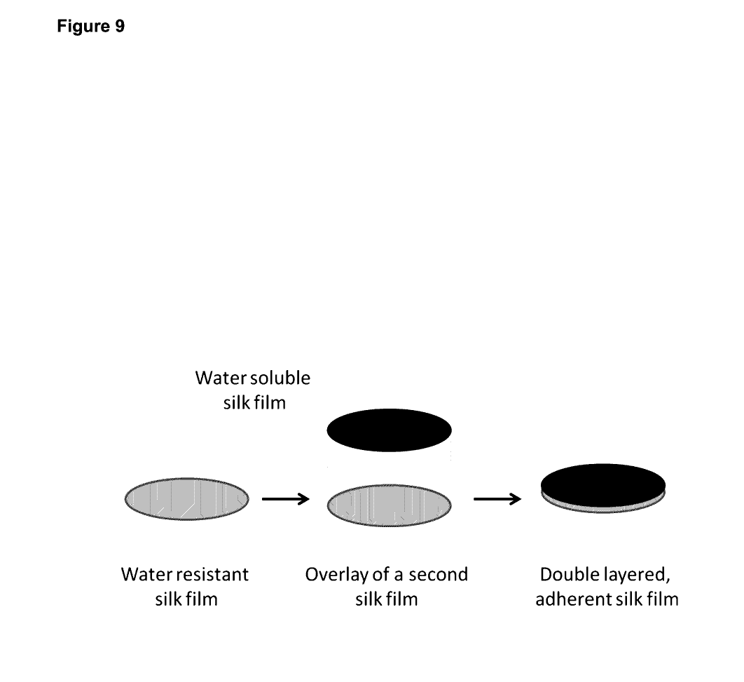

Silk fibroin solution (8% w/v, 1 ml) was cast on the bottom of two inverted 60 mm Petri dish and allowed to dry between 2-12 hours. Half of the films were then immersed for 2 hours in ethanol solution to induce beta-sheet formation. Subsequently, the ethanol treated films were rinsed with deionized water and repositioned on the molds. The remaining films (non-treated, water soluble silk films) were then deposited on top of the wet ethanol treated films and the double layered films was allowed to air dry for 2-12 hours (FIG. 9). Alternatively, a second layer of silk fibroin solution was deposited on top of the ethanol treated films, then allowed to dry, to yield the double layered self-adherent films.

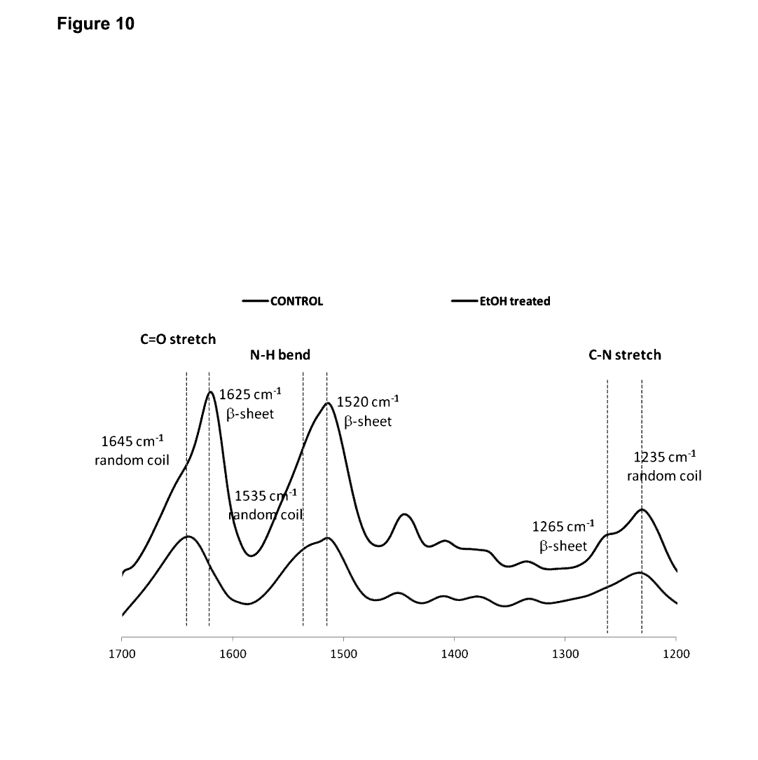

This Example 2 also made use of silk's natural ability to become water resistant via physical crosslinking. Through this process, the silk fibroin protein undergoes structural rearrangements to a beta-sheet rich conformation. Temperature, pH, ionic strength and treatment with polar agents such as alcohols are all factors known to induce such structural transitions. For the device made in this Example 2, beta sheet formation was induced via ethanol treatment (FIG. 10).

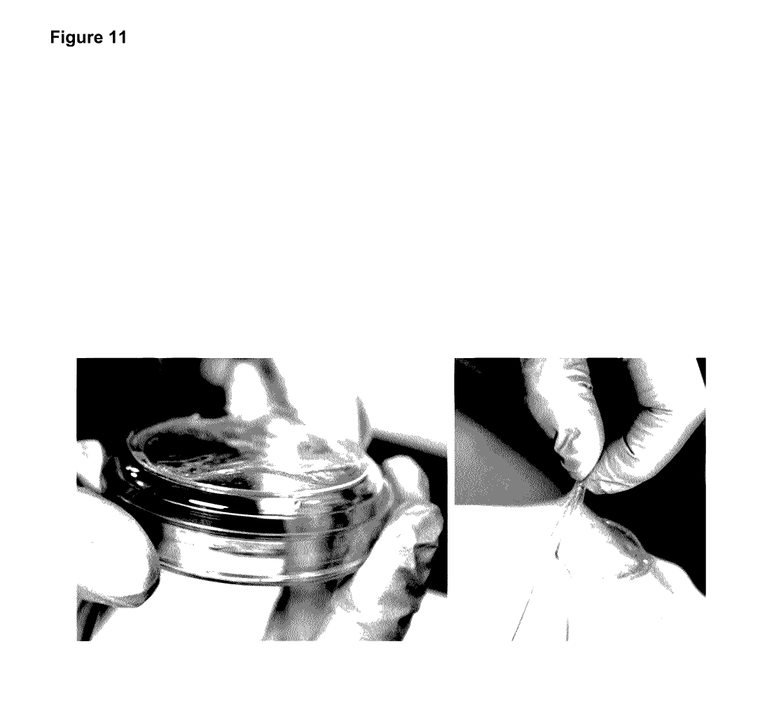

The devices made were smooth, double layered, self-adherent silk film consisting of a waterproof, physically crosslinked side and a water soluble, adherent side. The adhesiveness of the water soluble silk film is responsible for the cohesiveness of the double layered constructs as it intimately blended with the surface of the ethanol treated film. The dried device can be easily handled with dried gloves or hands. When applied to a wet or moist surface, the water soluble side of construct rehydrates and tightly adheres to the contact surface (FIG. 11). The ethanol treated side then provides a beta sheet rich, waterproof barrier.

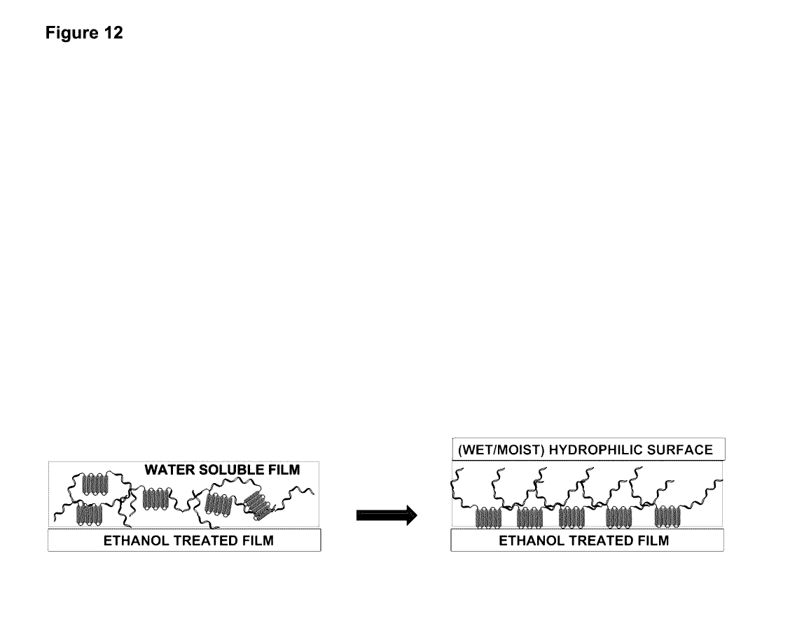

The film adherence mechanism probably implies structural rearrangements of the silk fibroin in which the hydrophilic regions of the protein get oriented toward and interact with the hydrophilic regions of the contact surface and analogously, the hydrophobic regions of the protein re-orient toward and interact with the hydrophobic, beta sheet rich interface of the ethanol treated silk film (FIG. 12).

The device can be used for example in: (a)—hemostasis (by attaching it or by juxtaposing it to bleeding blood vessels); (b)—wound dressing (by attaching it or by juxtaposing it to superficial wounds); (c)—burn dressings (by substituting skin grafts); (d)—small defect repair patch (by patching small defects such a tympanic membrane holes); (e)—tissue enforcing/supporting patch (by wrapping it against weakened tissues, i.e. cervix to prevent pre-term deliveries); or (f)—post-operative adhesion barrier (by attaching it to the affected tissue with the “sticky’ side, then the waterproof side would serve as a barrier to attachment to surrounding tissues). The versatility of this device is further highlighted by its transparency—which would enhance the ability to control the exact placement of the device; ease of sterilization—since it can be sterilely manufactured from autoclaved silk fibroin solution; control over the thickness and mechanical strength—since these parameters are dictated by the concentration of the silk solution used and the cast mold area; prolonged stability and cost effective manufacturing process.

### Example 3

### Use of Silk Medical Device in Abdominal Surgery

Briefly, a hernia is a bulge of intestine, another organ, or fat through the muscles of the abdomen, where tissue structure and function is lost at the load-bearing muscle, tendon and fascial layer. Thus, a hernia can occur when there is weakness in the muscle wall that allows part of an internal organ to push through. The silk medical device within the scope of the present invention can be used to assist in the repair of an inguinal (inner groin), incisional (resulting from an incision), femoral (outer groin), umbilical (belly button), or hiatal (upper stomach) hernia, using either an open or laproscopic technique. A ventral hernia is a type of abdominal hernia—it can develop as a defect at birth, resulting from incomplete closure of part of the abdominal wall, or develop where an incision was made during an abdominal surgery, occurring when the incision doesn't heal properly.

A silk medical device within the scope of the present invention can be used in both open and laparoscopic procedures to assist in the repair of a ventral hernia as follows: the patient lies on the operating table, either flat on the back or on the side, depending on the location of the hernia. General anesthesia is usually given, though some patients can have local or regional anesthesia, depending on the location of the hernia and complexity of the repair. A catheter is inserted into the bladder to remove urine and decompress the bladder. If the hernia is near the stomach, a gastric (nose or mouth to stomach) tube can be inserted to decompress the stomach. In an open procedure, an incision is made just large enough to remove fat and scar tissue from the abdominal wall near the hernia. The outside edges of the weakened hernial area are defined and excess tissue removed from within the area. The silk medical device is then applied so that it overlaps the weakened area by several inches (centimeters) in all directions. Non-absorbable sutures are placed into the full thickness of the abdominal wall. The sutures are tied down and knotted.

In the less-invasive laparoscopic procedure, two or three small incisions are made to access the hernia site—the laparoscope is inserted in one incision and surgical instruments in the others to remove tissue and place the silk medical device in the same fashion as in an open procedure. Significantly less abdominal wall tissue is removed in laparoscopic repair. The surgeon views the entire procedure on a video monitor to guide the placement and suturing of the silk medical device.

### Example 4

### Anti-Adhesive Silk Medical Devices

This Example 4 details the experiments we carried out to make and characterize various multi-component, multilayer or fused layers silk (or silk based) medical devices (“the device” or “the devices”). The devices we made are intended for implantation in humans or other mammals in a surgical or medical procedure, such as in a hernia repair surgical procedure, to assist in the repair and/or support of various soft tissues and prevent, or at least substantial reduce, adhesion formation onto the implanted devices or to adjacent tissues. Soft tissue can be tissues that connect, support, or surround other structures and organs of a mammalian (and in particular a human) body, such as tendons, ligaments, fascia, skin, fibrous tissues, fat, synovial membranes, connective tissue, muscles, nerves, blood vessels, as well as various soft tissue organs such as the breast.

The device is preferably made as a flat sheet. The device can comprise one layer or several layers of material. One layer or one side (i.e. the front) of the device is made of silk or is silk based, for example it is made of sericin extracted, knitted, silk fibroin yarn. When the device comprises only one layer of material the back or bottom side of the device has an adhesive property. When the device comprises two layers, the second layer on the opposite (i.e. the back side of the second layer) side of the device (attached to or fused the bottom side of the first layer) has the anti-adhesive property. The first layer can be and is preferably a silk fabric, such as SERI® Surgical Scaffold (available from Allergan, Inc., Irvine, Calif.). The anti-adhesive property of the second layer of a two layer device prevents the second layer once the device is abdominally implanted (or subsequent to the implantation of the device where the second layer is a sacrificial layer) facing the bowel, from attaching (or adhering) to the bowel.

The two layer devices are made by a multiple-step fabrication process, and can comprise a silk film or a silk fabric or mesh (a suitable and preferred silk fabric is SERI® Surgical Scaffold), as a first layer of the device, attached to a second layer which second layer forms an anti-adhesive barrier layer (when this version of the second layer faces the bowel the second layer is made of a biomaterial that does not promote cell attachment and proliferation).

Thus, as explained above the silk medical devices we developed have an anti-adhesive property either because the second layer does not promote cell attachment and proliferation or because the second layer is a sacrificial layer.

In this Example 4:
we carried out two in vitro cell screening assays to determine characteristics of various biomaterial substrates to use as the second layer of the device;
three devices comprising oxidized regenerated cellulose (“ORC”) as the second were made and characterized in vivo;
we made fourteen devices;
we tested in vitro devices which comprised a silk film (“SF”) and a hyaluronic acid (“HA”), an alginate (“ALG”), dextran sulfate (“DS”), a polyethylene glycol (“PEG”) or Pluronic® F127 (“F127”), and;
we made use of film casting and sponge casting technologies, as well an e-beam sterilization technique.

Table 1 shows the second layer materials (for a two or multi-layer device) we examined. Further details of each of these materials are provided in this Example 4.

TABLE 1
Anti-Adhesive Device Second Layer Materials Examined.

| | | | Anti-adhesive |
|---|---|---|---|
| Biomaterial | Structure | Source | properties |
| Hyaluronic acid (HA) | Anionic, linear, non- | Bacterial, avian or | Low cell attachment - |
| | sulfated polysaccharide | mammalian | due to hydrophilicity and |
| | | | negative charge |
| Dextran sulfate (DS) | Anionic, linear, highly | Bacterial(dextran)/ | Very low cell |
| | sulfated polysaccharide | synthetic (sulfated | attachment - due to |
| | | dextran) | negative charge |
| | | | inherent to high sulfate |
| | | | content |
| Alginate (ALG) | Anionic, linear, non- | Brown algae | Low cell attachment - |
| | sulfated polysaccharide | | due to hydrophilicity and |
| | | | negative charge |
| Polyethylene glycol | Hydrophilic, non-ionic | Synthetic | Low cell attachment - |
| (PEG) | polymer | | due to hydrophilicity |
| Pluronic ® F127 (F127) | Hydrophilic, non-ionic | Synthetic | Cytotoxic at higher |
| | polymer | | concentration (≧5% |
| | | | v/w), low cell |
| | | | attachment due to |
| | | | hydrophilicity |
| Oxidized regenerated | Anionic, linear, oxidized | Plants | Fibers supports cell |
| cellulose (ORC) | polysaccharide | | attachment because of |
| | | | local 3D topography, |
| | | | but ORC rapidly |
| | | | degrades and can be |
| | | | used as “sacrificial |
| | | | layer” |

### Selection of Anti-Adhesive Layer

An in vitro biomaterial screening experiment was carried out to:
rapidly evaluate the anti-adherence capacities of a large number of materials;
identify the most effective anti-adhesive material, and;
limit the number of devices tested in vivo.

This in vitro screening process involved the use of primary human fibroblasts (“the cells”, which are similar to the cells present at injury/surgery sites), and assessment of cell attachment, phenotype, proliferation and overall cell health, when cultured on different biomaterials. Thus our screening for suitable anti-adhesive material involved two main components: (a) the cells and (b) substrate biomaterials (the second layer). Additionally, the screening process was designed to allow the microscopical evaluation of the cells. For this purpose, we chose to prepare and evaluate the selected biomaterials (the second layer) as thin films, cast in wells of multi-well tissue culture plates. Although generally substrates can be presented to cells in a variety of physical forms such as gels, films, sponges, spheroids, etc., for our purpose it was considered that evaluation of cells on thin films was:
reflective of the cellular responses to formulation components
conveniently allow for microscopic cell phenotype evaluation because a light beam can easily pass through films
served the purpose of the pre-screening process by differentiating between substrate induced cellular changes

Materials (equivalent materials can also be used)
70% (v/v) ethanol solution (Fisher Scientific, cat #25467025)
Clorox bleach (Fisher Scientifics, cat #509387879)
Human dermal fibroblasts, adult (PCS-201-012, American Type Culture Collection (ATCC))
Fibroblast Basal Medium (ATCC, cat # PCS-201-030)
Fibroblast Growth Kit-Serum-free (ATCC, cat # PCS-201-040)
Fetal bovine serum (FBS) (ATCC, cat #30-2021)
Penicillin-Streptomycin-Amphotericin B solution (ATCC, cat # PCS-999-002)
Dulbecco's Phosphate Buffered Saline 1× (DPBS) (ATCC, cat #30-2200)
Eppendorf micropipetter set (Fisher Scientific, cat #13-684-251)
Filter top bottles (VWR, cat #154-0020)
Kimwipes (Fisher Scientific, cat #06-666-1A)
Cell culture flasks (T75 flasks, Fisher Scientific, cat #10-126-37)
Cell culture multi-well plates (24 well plates, Fisher Scientific, cat #08-772-4G)
Sterile serological pipettes, 1-50 ml (VWR, cat #89130)
Sterile aspirating pipettes, 2 ml (VWR, cat #414004-265)
Hemacytometer (Fisher Scientific, cat #02-672-5)
Cell dissociation reagent (Accutase) (Invitrogen, cat # A1110501)
Sterile conical tubes (50 ml) (Fisher Scientific, cat #07201332)
LIVE/DEAD® Viability/Cytotoxicity Kit, for mammalian cells (Invitrogen, cat # L3224)
Cell proliferation assay (Promega CellTiter 96 Aqueous One Cell Proliferation MTS Assay) (Fisher Scientific, cat # PR-G3580)
Sterile Petri dishes (60 mm diameter) (VWR)
Deionized water (Siemens (US Filter) RO/DI Water Purification System)
Parafilm@ Wrap (VWR)

Equipment
Humidified incubator (New Brunswick Excella E24R, VWR, Bridgeport, N.J.)
Laminar flow hood (SterilGard III Biohood; Allergan #0116)
Sterile surgical scissors (VWR)
Sterile forceps (VWR)

### Cell Culture

Primary human adult fibroblasts (HDFs) were obtained from the American Type Culture Collection (the ATCC, Manassas, Va. USA 20110) and cell cultures were initiated as per the ATCC instructions provided. Briefly, fibroblast specific cell culture media was prepared in the laminar flow hood, then the cell vial was thawed in a water bath at 37° C. for 1 minute. The cell suspension was then transferred to a T75 culture flask that contained 25 ml of culture medium. Cells were then incubated at 37° C. and 5% CO₂ and the culture medium was changed every 72 h until cells were needed for assays or became ˜80% confluent. When confluent, cells were subcultured in new flasks. Cells were propagated for a maximum of 6 passages throughout the duration of the study (HDFs have a maximum cycle of 10 propagations).

### Biomaterial (Second Layer) Casting Methods

Biomaterial films were prepared in the laminar flow hood from sterile filtered solutions, as described in Table 3. The solution concentrations used were chosen based on practical reasons:
The SF preparation process typically yields solutions with a silk concentration of 6-8% v/w. Higher of silk in the solution concentrations can be obtained by further processing, however, silk fibroin solution gels rapidly at concentrations over 8% v/v which make its handling difficult.
HA is a polymeric material with good aqueous solubility, however at concentrations over 2% w/v the solutions are highly viscous which makes their handling difficult
ALG is similar to HA, therefore we chose to use both these polysaccharides at 2% w/v
DS and PEG yielded low viscosity solutions at 10% w/v—higher concentrations would produce more viscous solutions that could not be sterile filtered
F127 has a critical gel transition temperature at 25° C. when used at 20% w/v, therefore this was chosen as stock concentration

Overall, the intent of this experiment was to have the working solution as concentrated as possible while keeping the viscosities at level that permitted pipetting, sterile filtration and transfer.

The volume ratios chosen for formulation screening were based on the need to obtain silk based solutions that would be physically crosslinkable via beta sheet interactions. This requirement ensures that the final scaffolds would not readily dissolved when placed in an aqueous environment and that no chemical crosslinkers are used in the process.

The film volumes (200 μl/well) was chosen based on the well area—this volume ensures uniform surface coverage while eliminating capillary tension effect (thicker film edges, thin film centers). It also provided minimal interference with the microscope light beam.

The crosslinking of the films prepared was performed with ethanol. For alginate based films, CaCl₂ was added to ethanol, as alginate gels in the presence of Ca²⁺ but is soluble in ethanol. HA, DS, PEG and F127 are also soluble in ethanol, however the SF crosslinking process entraps these macromolecules in the silk network even though some nano- and micro-scale heterogeneity arises in films because of the differential solubility of the components. The crosslinking solution volume (0.5 ml was chosen based on the volume of the tissue culture plate wells.

The biomaterial films were prepared as shown in Table 2.

TABLE 2
Summary of the biomaterial formulations screened for the development
of anti-adhesive devices and the casting method used.

| | Biomaterial formulation | | | | | | | | |
|---|---|---|---|---|---|---|---|---|---|
| | | | | | SF/DS | SF/DS | | | |
| | SF | SF/HA | ALG | SF/ALG | (HMW) | (LMW) | SF/PEG | SF/F127 | F127 |
| Starting | SF, | SF, | SF, | SF, | SF, | SF, | SF, | SF, | SF, |
| concentrations | 7.8- | 7.8-8.1% | 7.8-8.1% | 7.8-8.1% | 7.8-8.1% | 7.8-8.1% | 7.8-8.1% | 7.8-8.1% | 7.8-8.1% |
| (w/v) | 8.1% | HA, | ALG, | ALG, | DS, | DS, | PEG, | F127, | F127, |
| | | 2% | 2% | 2% | 10% | 10% | 10% | 20% | 10% |
| Volume ratios | NA | 1:1 | NA | 1:1 | 8:1 | 8:1 | 8:1 | 8:1 | NA |
| | | 2:1 | | 2:1 | | | | | |
| | | 3:1 | | 3:1 | | | | | |

| | |
|---|---|
| Casting | 200 μl/well of 24-well tissue culture plate |
| volume | |
| Drying | 18 h in laminar flow hood |

| | | | | |
|---|---|---|---|---|
| Crosslinking | 0.5 ml/well of | 0.5 ml/well of 1:1 | 0.5 ml/well of 90% EtOH for 30 min | NA |
| procedure | 90% EtOH for | v/v of 90% EtOH | | |
| | 30 min | and 0.5M CaCl₂ | | |
| | | in DPBS for 30 | | |
| | | min | | |

| | |
|---|---|
| Wash steps | 3X with sterile 1X DPBS, 0.5 ml/well |

### Film Surface Investigation

For the screening of biomaterials biological effects, the following biomaterials formulations were evaluated: SF; SF/HA (1:1; 2:1 and 3:1 v/v), ALG, SF/ALG (1:1; 2:1 and 3:1 v/v), SF/DS (8:1 v/v), SF/PEG (8:1 v/v), F127, SF/F127 (8:1 v/v). The 1:1 ratios were be well stabilized via SF crosslinking. However, higher SF content in the formulation conferred higher aqueous stability to the final formulation. For this we tested formulations with gradually increasing silk amounts. The surfaces of films cast as above were investigated microscopically at 100× magnification.

SF films had a smooth surface with cracks that originated most likely during the physical crosslinking process. SF/HA formulations showed a heterogeneous surface, most likely due to the fact that HA is insoluble in ethanol and tends to fall out of solution during the physical crosslinking process of silk. ALG (alginate) undergoes crosslinking in the presence of Ca²⁺. This caused the film to wrinkle and detach form the edges of the well. Due to the presence of the ethanol, needed to ensure similar treatment of all wells and also as an added measure of sterility, some amounts of alginate appeared to fall out of solution, similar to HA. SF/ALG formulations showed a heterogeneous surface, most likely due to the fact that ALG is insoluble in ethanol and tends to fall out of solution during the physical crosslinking process of silk. SF/PEG films were smooth due to the presence of PEG, which acts as a plasticizer and reduces the inter- and intra-molecular tension between silk molecules during the physical crosslinking process. The SF/DS films were smooth with some crater-like irregularities, most likely generated by the differences in solubility between SF and DS. F127 poloxamer, at concentrations of 15% w/v and above, gels at room temperature and showed a smooth surface. F127 is however soluble in ethanol and some material most likely washed off during the ethanol treatment. The SF/F127 films surfaces were heterogeneous. F127 was expected to act as plasticizer, however the differences in solubility between SF and F127 are probably the cause for the observed surface irregularities.

### Cell Attachment Evaluation

In the context of the development of a device with or with a layer of the device that has an anti-adhesive property, cell attachment was evaluated as a primary indicator of the device or the layer's anti-adhesive efficiency (the lower the cell attachment the better the anti-adhesive properties of the biomaterial). Primary human dermal fibroblasts (adult, HDF) passage 5 were cultured on the biomaterial films made at a density of 2×10⁵ cells/ml corresponding to 5000 cells/well in 250 μl culture medium. The cell seeding concentration was chosen based on the culture surface area, the HDF proliferation pattern observed during cell culturing and assay duration (slow proliferating cells would be seeded at high numbers, while fast proliferating cells would be seeded at low numbers to avoid contact inhibition issues at longer than 24 h (hour) assay time points). Cell morphology and attachment were visually assessed after 24 hours and 6 days incubation.

On the tissue culture plate (“TCP”) control, HDFs show the fibroblast specific, spindle-shaped morphology, both at 24 h and at 6 days. The 6 day data revealed a healthy cell phenotype with good proliferation. This data set represented our positive control: because the TCP surface is designed to support and promote cell attachment and viability (ATCC animal cell culture guide). For all our anti-adhesive device formulations, we targeted lower cell attachment than that observed on the TCP.

The 24 h data images were representative for the observed phenotypes on the entire film surface and revealed atypical fibroblast phenotypes on all formulations, with SF, SF/PEG and SF/DS films still induced elongated, somewhat spindle-like phenotypes, but the overall cell morphology was different than on TCP showing that cell attachment was impaired, as desired. SF/HA and SF/ALG prevented cell attachment to the point where cells were rounded and clustered together.

The 6 day data revealed further cellular changes. On SF, cells were covering the surface unevenly and were anchored to few attachment points most probably corresponding to cracks in the film surface. This showed that SF enhanced anti-adhesive properties compared to the TCP. On SF/HA some cell spreading was noticed, however the surface coverage appeared to be less than the TCP control, as estimated via microscopic evaluation. SF/ALG and SF/DS prevented cell spreading and a few rounded cell clusters were observed on the surface of these biomaterials.

Significantly, all the biomaterial formulations we made and evaluated showed decreased cell attachment and surface coverage as compared to the TCP control. This showed that each of the chosen second layer materials evaluated can be used as the anti-adhesive layer of the device. It is important to note that although referred to above as a second layer, the biomaterials used were in fact fused to the silk film layer (SF) used. The anti-adhesive second layer can alternately be attached or fused to a first layer which is in the form of a silk fabric or a silk mesh.

### Cell Viability Assay

The cell attachment assay offered a visual assessment of the desired anti-adhesive/cell-repellant properties of different biomaterial formulations. In addition to this feature, it was important to evaluate the actual effects of SF and the second layer materials (“additives” or “biomaterials”) on cell viability.

The cytocompatibility of biomaterials was evaluated after 48 h and 6 day incubation period. For this, a LIVE/DEAD cytotoxicity kit was used. This kit has two components: fluorescein (green fluorescence)—a dye that binds to the membrane of intact, live cells; and ethidium homodimer (red fluorescence)—a nucleic acid specific dye that binds to the nucleus of damaged/dead cells, but cannot permeate the membrane of healthy cells.

Post-plating, the duration of cell attachment is dependent on the cell type and substrate, and might take up to 24 h to complete (ATCC animal cell culture guide). Therefore, the 48 h time point was chosen as it is the earliest point that would allow evaluation of substrate related cytotoxic effects after cell attachment has occurred. The 6 day time point was chosen to evaluate the longer term cytocompatibility of the substrates as the potential effects of additive leaching was expected to be detectable at this time point (after 6 days, cells on some substrates reached confluence, therefore we chose not to investigate later time points). Cell viability on TCP was used as the positive control.

At both time points all the second layer films we had prepared showed minimal cytotoxic effects, with cell viability exceeding 95% as determined by microscopic evaluation. The only second layer material the appeared to induce cell death was pluronic F127 as stand-alone formulation. We also noted that based on the substrate (second layer) formulations, the cells had different phenotypes—a more rounded appearance indicative of lower attachment while a spindle-like phenotype was indicative of better attachment, comparable to the control.

In summary, the cytocompatibility assay showed that all the tested second layers were cytocompatible and did not induce cell death. This showed that SF and the tested additives (the second layer materials) can be used in the device.

### Cell Proliferation Assay

Another method we used to evaluate the affinity of cells to a surface and the cell-substrate interaction was to perform a cell proliferation assay (MTS assay). This assay relied on the cell mediated enzymatic reduction of a soluble methyl tetrazolium salt (MTS) to its reduced, colored format, therefore eliminating the possibility of any artifacts or false positives. This enzymatic reduction process gave a direct correlation between the number of living cells on a surface and the color intensity of the reduced MTS.

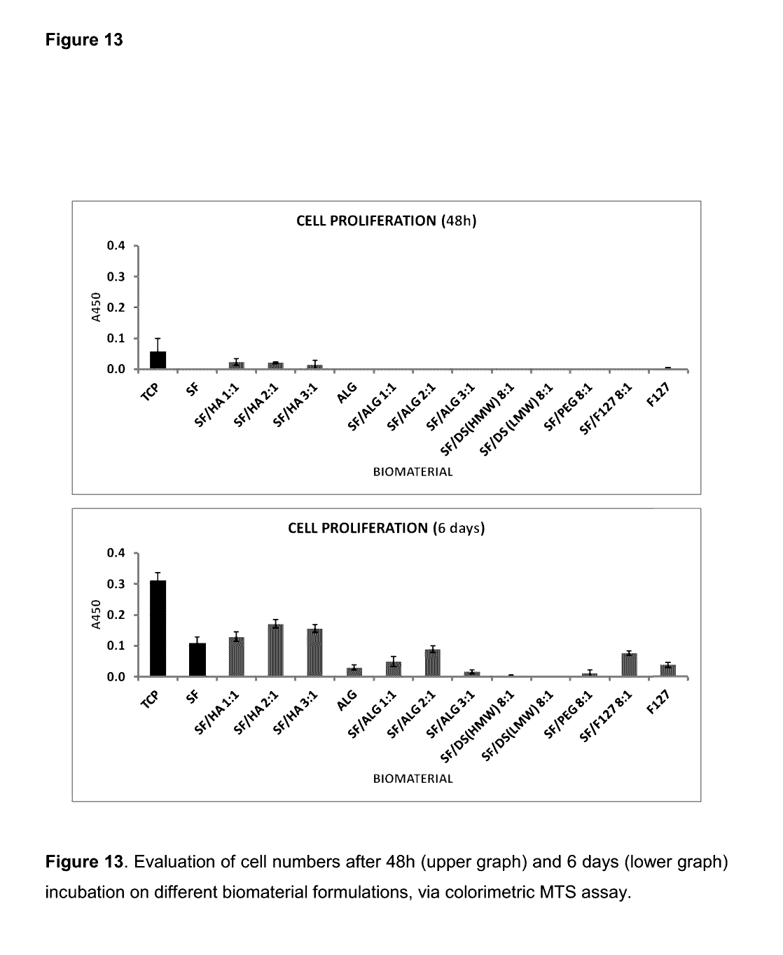

The cell numbers present on the screened biomaterial surfaces were evaluated at 48 h and 6 days post incubation. The 48 h time point was chosen as an early indicator of cellular affinity to the films, however the 6 day readings were more representative since the assay is sensitive to the overall cell number and yields better results for higher cell densities, such as those observed at later incubation time points (FIG. 13). At 48 h, the A450 values were lower than those observed at 6 days. This was consistent with a lower initial cell number/well correlating with attachment differences. As attached cells proliferate and their number increases in the test well, when assayed, the intensity of the colorimetric reagent increases and this translates into higher A450 values, as seen at day 6. Nevertheless, the 48 h and 6 day data trends correlated well and supported earlier observations indicating that all screened biomaterial formulations (the second layer) showed anti-adhesive potential as cell attachment was ≦50% lower on all formulations compared to the TCP control.

### Device Formulation Screening

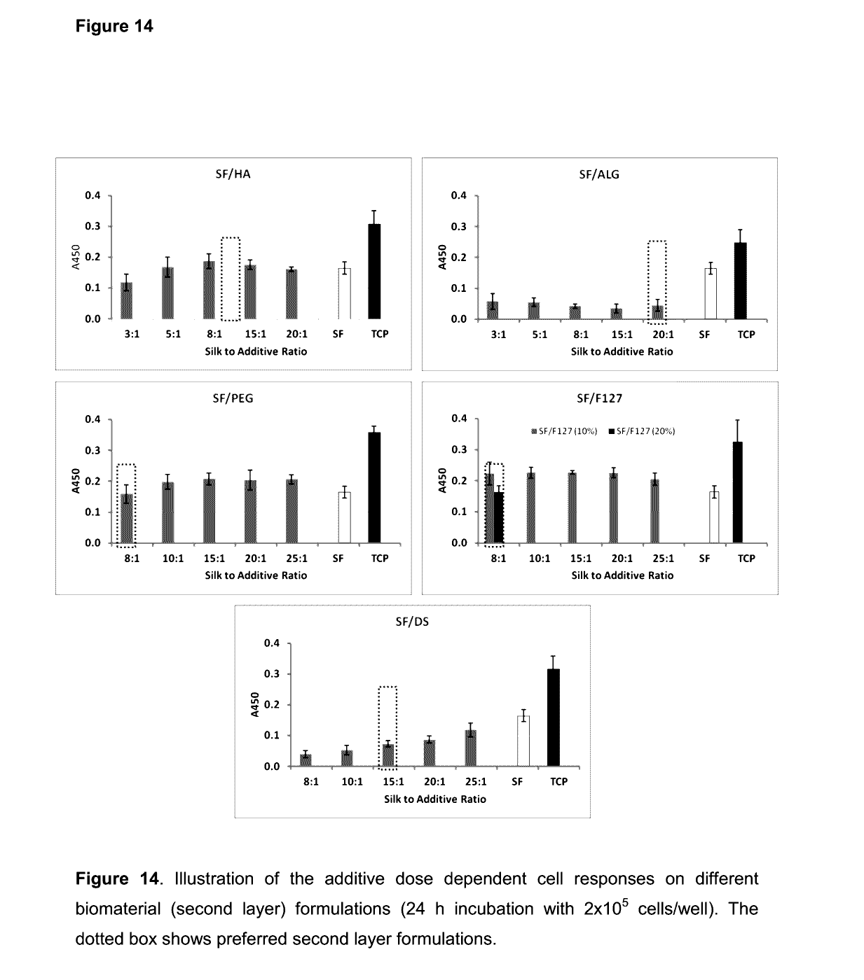

The results of the aforementioned cell screening data showed that the chosen formulations (second layer materials) had the desirable anti-adhesive feature. We wished to minimize the amount of additives (second layer material) in order to maintain the silk (first layer) characteristic physical crosslinking and to avoid any potential or unknown negative interactions these might cause so we therefore additionally screened the formulations (second layer materials) with increased silk content. For SF as the first layer with five different second layer materials, Table 3 summarizes the five tested SF/additive formulations and the results are illustrated in the five FIG. 14 bar graphs.

TABLE 3
Summary of SF/additive formulations tested in order to minimize the
amount of additives while maintaining the biological properties.

| Biomaterial | | | | | |
|---|---|---|---|---|---|
| formulation | SF/HA | SF/ALG | SF/DS | SF/PEG | SF/F127 |
| Volume ratios | 3:1 | 3:1 | 8:1 | 8:1 | 8:1 |
| (v/v) | 5:1 | 5:1 | 10:1 | 10:1 | 10:1 |
| | 8:1 | 8:1 | 15:1 | 15:1 | 15:1 |
| | 15:1 | 15:1 | 20:1 | 20:1 | 20:1 |
| | 20:1 | 20:1 | 25:1 | 25:1 | 25:1 |

The biomaterial (second layer) films were prepared as described above. HDF cells were plated at a density of 2×10⁵ cells/well and incubated for 24 h before assayed for cell number (MTS assay). A higher cell seeding density was chosen for this short duration assay in order to maximize assay sensitivity. For SF/HA formulations, the data showed that the 3:1 volume ratio yielded the best biological outcome (equivalent to the lowest cell concentration) and that decreasing the HA amount in the formulation can increase cell adhesion. However, during the device casting, the sponge surface appeared to shed upon rubbing. Therefore, a 10:1 SF to HA ratio was chosen for device evaluation since it was the highest HA containing formulation that yielded a robust sponge surface. For SF/ALG the 20:1 ratio yielded similar biological effects to higher ALG ratios, therefore devices were prepared with 20:1 SF to ALG. For SF/PEG the 8:1 ratio produced the best biological outcomes, therefore devices were prepared with 8:1 SF to PEG. For SF/F127, when F127 was used at 10% w/v concentration, the cell adherence was similar on all formulations. When 20% w/v F127 was used, at an 8:1 volume ratio, the cell repellent effects were more pronounced than for its 10% w/v counterpart. However, because of cytotoxicity concerns as evident from cells seeded pure F127, devices were made with 8:1 SF to F127 (10% w/v). For SF/DS, the tested formulations elicited a clear dose response, with cell attachment being the lowest in the presence of the highest amount of DS (corresponding to the 8:1 SF/DS formulation). In our assays DS showed good biocompatibility. Since all ratios were more anti-adhesive than the SF control, we made devices with a 15:1 SF to DS ratio, to minimize the amount of additive but still maintain the significantly increased anti-adherent properties.

### Device Preparation

Materials
SERI® Surgical Scaffold (Allergan), a knitted silk mesh.
Silk fibroin solution (prepared as set forth in International Patent Application WO/2010/123945; see paragraph [0011], ibid.)
Silk yarn (9-filament)
HA, high molecular weight (HMW) (intrinsic viscosity 2.84 m³/kg)
HA, low molecular weight (HMW) (intrinsic viscosity 0.41 m³/kg)
DS (HMW) (Sigma, cat #67578, lot #, MW 200 kDa)
DS (LMW) (Sigma, cat #42867, lot # BCBK1677V, MW 40 kDa)
ORC as Surgicel® SNoW absorbable hemostat (ref #2083, lot ELB5821, Ethicon)
ORC as Surgicel® NuKnit absorbable hemostat (ref #1946, lot #3650291, Ethicon)
ORC as Surgicel® Fibrillar absorbable hemostat (ref #1963, lot #3653407, Ethicon)
ORC as Surgicel® Original absorbable hemostat (ref #1952, lot #3649196, Ethicon)
ALG (NovaMatrix ProNova, SLG100, lot #271108/3)
PEG (Alfa Aesar, cat #43443, lot # J04Y009, MW 8 kDa)
F127 (Sigma, cat # P2443, lot # SLBC8439V). F127 and Pluronic F127 is an ethylene oxide, propylene oxide block copolymer.
Conical tubes (50 ml, Fisher Scientific, cat #339653)
Petri dishes (100 mm diameter, VWR, cat #25384-342)
Square dishes (110 mm×15 mm, VWR, 100501-176)
OmniTray (Nunc 86×128 mm, Fisher Scientific, cat #242811)
Cleanroom Wipes (Berkshire, DR670.1212.20)
Ethanol (100%, Fisher Scientific, cat #50-980-460)

Equipment
Freezer (−80° C.) (VWR Model 5708)
Lyophilizer (VirTis Model Benchtop K)
Water bath (Thermo Fisher Precision, Fisher Scientific)
Laminar flow hood (SterilGARD II Advance, The Baker Company)
Incubator (Forma Scientific Model 3326, Fisher Scientific)
Centrifuge (Eppendorf Model 5804, Fisher Scientific)
Refrigerator (NorLake Scientific, Fisher Scientific)
Bright field microscope (Leica Model DMI3000B)
Vacuum oven (WVR Model 1410)
Stainless steel scissors (VWR)
Tweezers (WVR)
Lead rings (Fisher Scientific, cat #22-260-103)
Sewing machine (JUKI Corporation Model DDL-5530N)
Sterilizations pouches metallic (MPPE) (PeelMaster Medical Packaging, item #1854-024)
Sterilization pouches foil (PPFP) (PeelMaster Medical packaging, item #1854-024)
Pouch sealer (Accu-Seal Model 630)
Self-seal sterilization pouches (VWR, cat #89140-800)
Flatiron (Black & Decker, cat # A5870 Type I)

### General Considerations

The devices were with the second layers selected based on the results set forth above. Depending on the properties, some formulations were prepared as films and some as sponges. Biomaterial (second layer) mixes that yielded homogeneous formulations, with good pliability were cast as films (SF/PEG and SF/F127), while based on the same considerations, sponges appeared to be a more suitable option for heterogeneous materials such as SF/HA, SF/ALG and SF/DS. Films fused with silk mesh were easily sutured as films and were transparent, however the sponges fused with silk mesh appeared to have more robustness during handling (film devices can delaminate when crumpled in hand, while crumpling was not an issue with the sponges made). For certain devices made additional composition adjustment were made to improve their processability.

For sterilization, some devices were processed dry, with ethylene oxide, while other were processed moist, with e-beam sterilization. The intent was to sterilize all samples dry, however, with film and certain sponges, drying caused curling and cracking of the scaffold. Based on this, we chose to process films and sponges moist, sealed in pouches with moisture barrier. Since ethylene oxide cannot be used with such pouches, samples were processed via e-beam treatment. The sterilization of all devices was performed with standard sterilization cycles and parameters and no additional sterility control was performed on any of the devices.

### Process

### Device 1

Description:

SERI® Surgical Scaffold fused with Surgicel® SNoW (ORC) (6×6 cm)

Execution:

Sterile SERI® Surgical Scaffolds were cut in the laminar flow hood with sterile stainless steel scissors into 6×6 cm squares. Similarly, Surgicel® SNoW were cut in the laminar flow hood with sterile stainless steel scissors into 6×6 cm squares. Autoclaved silk fibroin solution (c=7.5% w/v) was used to mount Surgicel SNoW onto the mesh. Specifically, 2 ml of silk solution were added to the lid of a sterile 100 cm Petri dish and was evenly spread with a sterile pipette tip. The mesh was then placed in the dish until its surface was uniformly wet. The mesh was transferred onto a Surgicel SNoW square and pressed down with sterile tweezers for 1 minute. All assembled devices were then allowed to dry for 1 h in the laminar flow hood then ethanol treated (100% v/v) for 30 minutes. The ethanol was then allowed to evaporate and devices were individually washed with 150 ml of sterile PBS. The washing step was done by using a vacuum filtration flask—the device was laid flat on the top filter, the filter was connected to vacuum and 15 ml of PBS were poured onto the device. The vacuum helped remove most of the PBS from the devices. The prototypes were then further dried on the laminar flow hood for 12 h in partially covered sterile rectangular plates (OmniTrays).

Sterilization:

These devices were assembled in the laminar flow hood from sterile starting materials. No additional sterilization was performed.

Packaging/Storage:

Devices prepared as above were places in autoclaved containers and covered with sterile PBS. They were kept under ambient conditions for 24 h before use.

Testing:

Device 1 was used as “wet lab” material to consolidate the surgical procedure. No additional testing was performed.

Observations:

Partial delamination of the two layers was observed for some of the device 1 samples made.

### Device 1A

Description:

SERI® Surgical Scaffold sewn with Surgicel® SNoW (ORC) (6×6 cm)

Execution:

Non-sterile SERI® Surgical Scaffolds were cut with sterile stainless steel scissors into 6×6 cm squares. Similarly, Surgicel® SNoW were cut under non-sterile conditions with stainless steel scissors into 6×6 cm squares. For each device one mesh square was sewn with a sewing machine to one SNoW square by using extracted 9-filament silk yarn.

Sterilization:

Devices were placed in self-sealing pouches and ethylene oxide (EO) sterilized.

Packaging/Storage:

Devices 1A prepared were placed in self-sealing sterilization pouches, were EO sterilized then aerated for at least 3 day prior use. During the aeration period, devices were kept under environmental conditions.

Testing:

Prototype 1A was tested in vivo

### Device 2

Description:

SERI® Surgical Scaffold fused with Surgicel® NuKnit (ORC) (6×6 cm)

Execution:

Sterile SERI® Surgical Scaffolds were cut in the laminar flow hood with sterile stainless steel scissors into 6×6 cm squares. Similarly, Surgicel® NuKnit were cut in the laminar flow hood with sterile stainless steel scissors into 6×6 cm squares. Autoclaved silk fibroin solution (c=7.5% w/v) was used to mount Surgicel NuKnit (patterned side up) onto the mesh. Specifically, 1 ml of silk solution was added to the lid of a sterile 100 cm Petri dish and was evenly spread with a sterile pipette tip (the amount of silk used for fusing the two layers was reduced to 1 ml in this case as dipping of the mesh into 2 ml of silk caused wetting of NuKnit and impaired the fusion of the layers). The mesh was then placed in the dish until its surface was uniformly wet. The mesh was transferred onto a NuKnit square and pressed down by rolling the bottom of a Petri dish on its side. All assembled devices were then allowed to dry for 1 h in the laminar flow hood then ethanol treated (100% v/v) for 30 minutes. The ethanol was then allowed to evaporated and devices were individually washed with 150 ml of sterile PBS. The washing step was done by using a vacuum filtration flask—the device was laid flat on the top filter, the filter was connected to vacuum and 15 ml were poured onto the device. The vacuum helped remove most of the PBS from the devices. The prototypes were then further dried on the laminar flow hood for 12 h in partially covered sterile rectangular plates (OmniTrays).

Sterilization:

These devices were assembled in the laminar flow hood from sterile starting materials. No additional sterilization was performed.

Packaging/Storage:

Devices prepared as above were places in autoclaved containers (see image above) and covered with sterile PBS. They were kept under ambient conditions for 24 h before use.

Testing:

Prototype 2 was used as “wet lab” material to consolidate the surgical procedure. No additional testing was performed.

Observations:

Partial delamination of the two layers was observed for some of the Device 2 samples.

### Device 2A

Description:

SERI® Surgical Scaffold sewn with Surgicel® NuKnit (ORC) (6×6 cm)

Execution:

Non-sterile SERI® Surgical Scaffolds were cut with sterile stainless steel scissors into 6×6 cm squares. Similarly, Surgicel® NuKnit were cut under non-sterile conditions with stainless steel scissors into 6×6 cm squares. Based on the tightly knit pattern of the ORC, the sacrificial layer of Prototype 2A is expected to degrade slower than that of Prototype 1A. For each device one mesh square was sewn with a sewing machine to one NuKnit square (patterned side up) by using extracted 9-filament silk yarn.

Sterilization:

Devices were placed in self-sealing pouches and ethylene oxide (EO) sterilized.

Packaging/Storage:

Devices prepared as above were placed in self-sealing sterilization pouches, were EO sterilized then aerated for at least 3 day prior use.

Testing:

Device 2A was tested in vivo.

### Device 3

Description:

SERI® Surgical Scaffold sewn with Surgicel® Fibrillar (2 sheets) and Surgicel® Original (ORC) (6×6 cm)

Execution:

Non-sterile SERI® Surgical Scaffolds were cut with sterile stainless steel scissors into 6×6 cm squares. Similarly, Surgicel® Fibrillar and Surgicel® Original were cut under non-sterile conditions with stainless steel scissors into 7×7 cm squares. For each device one mesh square was sewn with a sewing machine to two sheets of Surgicel® Fibrillar and topped with one layer of Surgicel® Original by using extracted 9-filament silk yarn. The combination of the two ORC materials ensured a thicker sacrificial layer that could potentially degrade at a slower rate than that of Device 1A or Device 2A. The assembled device was then trimmed to a size of 6×6 cm.

Sterilization:

Devices were placed in self-sealing pouches and ethylene oxide (EO) sterilized.

Packaging/Storage:

Devices prepared as above were placed in self-sealing sterilization pouches, were EO sterilized then aerated for at least 3 day prior use.

Testing:

Device 2A was tested in vivo.

Device 4 and Device 5 were silk based control devices (SBR-202 and SERI 3D).

### Device 6

Description:

SERI® Surgical Scaffold fused with SF/PEG film (6×6 cm)

Execution:

Non-sterile SERI® Surgical Scaffolds were cut with stainless steel scissors into 6×6 cm squares. Separately, silk fibroin solution (c=8.1% w/v) was mixed with PEG (c=10% w/v) in a 8:1 volume ratio then the mix was homogenized by pipetting up and down. The solution (8 ml) was cast in 10 cm square Petri dish bottoms and dried in the vacuum oven for 18 h. Dried films in dishes were then treated with 6 ml of ethanol (100% v/v) for 5 min. Films were then removed from dishes and briefly hydrated by a 5 second dip in deionized water, followed by 5 second dip in 90% v/v ethanol. Subsequently, films were placed face down (the side that was exposed to air during drying) and stretched on the lid of a 100 mm Petri dish then allowed to dry flat with a Petri dish bottom and a lead ring sitting on top.

Silk fibroin solution (c=8.1% w/v) was used to mount the mesh onto the films (“c” means “concentration”). Specifically, 2 ml of silk solution were added to the lid of a sterile 100 cm Petri dish and were evenly spread with a sterile pipette tip. The mesh was placed in the dish until its surface was uniformly wet. The mesh was then added onto the dried film and smoothed down with gloved fingers to ensure uniform surface attachment. The constructs were dried flat with the bottom of a 100 mm Petri dish and a lead ring resting on top for 15 minutes. The devices were then placed in 90% ethanol for 10 minutes, blotted, then placed in deionized water for 5 minutes for first wash. After the first wash, films were trimmed down to the size of the 6×6 cm mesh and then placed into second wash for 5 minutes. The devices were washed one more time then pouched.

Sterilization:

the devices were placed in metallized peelable polyester polyethylene film (MMPE) and paper polyethylene foil polyethylene barrier (PPFP) pouches, sealed using Accu-Seal Sealer Model 630, and e-beam sterilized.

Packaging/Storage:

The devices prepared as above were placed in PPFP pouches, heat sealed and e-beam sterilized. The devices were then kept in pouches and stored.

Testing:

The devices in both MMPE and PPFP pouches were examined two weeks after sterilization for overall integrity, delamination, pliability and suturability.

Observations:

Films remained moist during and after sterilization. Devices appeared to have good pliability but when crumpled in hand, the films separated from the mesh. Devices were easy to suture through as the films are transparent and mesh pores are clearly visible.

### Device 7

Description:

SERI® Surgical Scaffold fused with SF/F127 film (6×6 cm)

Execution:

Non-sterile SERI® Surgical Scaffolds were cut with stainless steel scissors into 6×6 cm squares. Separately, silk fibroin solution (c=8.1% w/v) was mixed with F127 (c=10% w/v) in a 8:1 volume ratio then the mix was homogenized by pipetting up and down. The solution (8 ml) was then cast in 10 cm square Petri dish bottoms and dried on the bench top for 26 h.

Silk fibroin solution (c=8.1% w/v) was used to mount the mesh onto the films. Specifically, 2 ml of silk solution were added to the lid of a sterile 100 cm Petri dish and were evenly spread with a sterile pipette tip. The mesh was placed in the dish until its surface was uniformly wet. The mesh was then added onto the dried film and smoothed down with gloved fingers to ensure uniform surface attachment. The construct was dried flat on bench top for 45 minutes, then placed in 90% ethanol for 45 minutes. Subsequently, the prototype was placed in 1 L deionized water for 1 h then dried on bench top. The drying process caused films to shrink, curl and detach from the mesh. Out of seven constructs prepared, two appeared well fused and smooth and were sent for sterilization.

Sterilization:

Devices were placed in self-sealing pouches and ethylene oxide (EO) sterilized.

Packaging/Storage:

Sterile devices were kept in pouches for under environmental conditions

Testing:

Pouches were opened in the laminar flow hood and prototypes were assessed for integrity and biological properties in vitro.

Observations:

Devices remained intact during and after sterilization and when tested for cell adherence, the results were comparable to the pre-sterilization data indicating that EO sterilization did not alter the device's biological properties.

### Device 7A

Description:

SERI® Surgical Scaffold fused with SF/F127 film (6×6 cm)

Execution:

Non-sterile SERI® Surgical Scaffolds were cut with stainless steel scissors into 6×6 cm squares. Separately, silk fibroin solution (c=8.1% w/v) was mixed with F127 (c=10% w/v) in a 8:1 volume ratio then the mix was homogenized by pipetting up and down. The solution (6 ml) was then cast in 10 cm square Petri dish bottoms and dried in the vacuum oven for 18 h. Dried films in dishes were then treated with 6 ml of ethanol (100% v/v) for 5 min. Films were then removed from dishes and briefly hydrated by a 5 second dip in deionized water, followed by a 5 second dip in 90% v/v ethanol. Subsequently, films were placed face down (the side that was exposed to air during drying) and stretched on the lid of a 100 mm Petri dish then allowed to dry flat with a Petri dish bottom and a lead ring sitting on top.

Silk fibroin solution (c=8.1% w/v) was used to mount the mesh onto the films. Specifically, 2 ml of silk solution were added to the lid of a sterile 100 cm Petri dish and were evenly spread with a sterile pipette tip. The mesh was placed in the dish until its surface was uniformly wet. The mesh was then added onto the dried film and smoothed down with gloved fingers to ensure uniform surface attachment. Constructs were dried flat with the bottom of a 100 mm Petri dish and a lead ring resting on top for 15 minutes. Prototypes were then placed in 90% ethanol for 10 minutes, blotted, then placed in deionized water for 5 minutes for first wash. After the first wash, films were trimmed down to the size of the 6×6 cm mesh and then places into second wash for 5 minutes. Prototypes were washed one more time then pouched.

Sterilization:

Devices were placed in metallized peelable polyester polyethylene film (MMPE) and paper polyethylene foil polyethylene barrier (PPFP) pouches, sealed using Accu-Seal Sealer Model 630, and e-beam sterilized

Packaging/Storage:

Devices prepared as above were placed in PPFP pouches, heat sealed and e-beam sterilized. Devices were then kept in pouches and stored.

Testing:

Devices in both MMPE and PPFP pouches were examined two weeks after sterilization for overall integrity, delamination, pliability and suturability.

Observations:

Films remained moist during and after sterilization. Devices had good pliability but when crumpled in hand, the films separated for from the mesh. Devices were easy to suture through as the films are transparent and mesh pores are clearly visible.

### Device 8

Description:

SERI® Surgical Scaffold fused with SF

Execution:

Non-sterile SERI® Surgical Scaffolds were cut with stainless steel scissors into 6×6 cm squares. Separately, silk fibroin solution (c=8.1% w/v) was mixed with HA (LMW, c=2% w/v) in a 3:1 volume ratio then the mix was homogenized by pipetting up and down. To obtain a sponge-like biomaterial, the solution (15 ml) was then cast in OmniTray lids and put into the −80° C. freezer for two hours. Frozen samples were lyophilized for 24 hours to dry, then treated with 15 ml of ethanol (100% v/v) for 45 min. Sponges were then removed from the tray, the edges were cut off, then were returned to the tray for an additional 30 minutes ethanol incubation. Subsequently, sponges were dried flat covered with OmniTray lids and lead rings.

Silk fibroin solution (c=8.1% w/v) was used to mount the mesh onto the films. Specifically, 2 ml of silk solution were added to the lid of a sterile 100 cm Petri dish and were evenly spread with a sterile pipette tip. The mesh was placed in the dish until its surface was uniformly wet. The mesh was added onto the dried sponge (to the side that contacted the tray while freezing) then smoothed down with gloved fingers to ensure uniform surface attachment. Prototypes were dried for 1 hour, then placed in 90% ethanol for 30 minutes and blotted. Subsequently, constructs were placed in deionized water for 5 minutes for first wash. After the first wash, sponges were trimmed down to the size of the 6×6 cm mesh and then placed into second wash for 5 minutes. Prototypes were washed one more time then pouched.

Sterilization: Devices were placed in MMPE and PPFP pouches, sealed and e-beam sterilized.

Testing: Devices in both MMPE and PPFP pouches were examined two weeks after sterilization for overall integrity, delamination, pliability and suturability.

Observations: Devices remained moist during and after sterilization. The devices had good pliability and did not delaminate when crumpled in hand. However, the sponge side appeared to shed when rubbed with gloved hands. The devices were easy to suture through.

Device 8A

Description:

SERI® Surgical Scaffold fused with SF/HA sponge (6×6 cm)

Execution:

Non-sterile SERI® Surgical Scaffolds were cut with stainless steel scissors into 6×6 cm squares. Separately, silk fibroin solution (c=8.1% w/v) was mixed with HA (LMW, c=2% w/v) in a 10:1 volume ratio then the mix was homogenized by pipetting up and down. To obtain a sponge-like biomaterial, the solution (15 ml) was then cast in OmniTray lids and put into the −80° C. freezer for two hours. Frozen samples were lyophilized for 24 hours to dry, then treated with 15 ml of ethanol (100% v/v) for 45 min. Sponges were then removed from the tray, the edges were cut off, then were returned to the tray for an additional 30 minutes ethanol incubation. Subsequently, sponges were dried flat covered with OmniTray lids and lead rings. The sponge can be viewed as a particular type of film (a sponge like film).

Silk fibroin solution (c=8.1% w/v) was used to mount the mesh onto the films. Specifically, 2 ml of silk solution were added to the lid of a sterile 100 cm Petri dish and were evenly spread with a sterile pipette tip. The mesh was placed in the dish until its surface was uniformly wet. The mesh was added onto the dried sponge (to the side that contacted the tray while freezing) then smoothed down with gloved fingers to ensure uniform surface attachment. Prototypes were dried for 1 hour, then placed in 90% ethanol for 30 minutes and blotted. Subsequently, constructs were placed in deionized water for 5 minutes for first wash. After the first wash, sponges were trimmed down to the size of the 6×6 cm mesh and then placed into second wash for 5 minutes. The devices were washed one more time then pouched.

Sterilization:

Devices were placed in MMPE and PPFP pouches, sealed and e-beam sterilized.

Packaging/Storage:

Devices were then kept in pouches and stored in a plastic bin under environmental conditions.

Testing:

Devices in both MMPE and PPFP pouches were examined two weeks after sterilization for overall integrity, delamination, pliability and suturability.

Observations:

The devices remained moist during and after sterilization, had good pliability and did not delaminate when crumpled in hand. This specific SF/HA formulation yielded sponges that did not shed when rubbed with gloved hands and the devices were easy to suture through.

### Device 9

Description:

SERI® Surgical Scaffold fused with SF/DS sponge (6×6 cm)

Execution: Non-sterile SERI® Surgical Scaffolds were cut with stainless steel scissors into 6×6 cm squares. Separately, silk fibroin solution (c=8.1% w/v) was mixed with DS (LMW, c=10% w/v) in a 15:1 volume ratio then the mix was homogenized by pipetting up and down. To obtain a sponge-like biomaterial the solution (15 ml) was then cast in OmniTray lids and put into the −80° C. freezer for two hours. Frozen samples were lyophilized for 24 hours to dry, then treated with 15 ml of ethanol (100% v/v) for 45 min. Sponges were then removed from the tray, the edges were cut off, then were returned to the tray for an additional 30 minutes ethanol incubation. Subsequently, sponges were dried flat covered with OmniTray lids and lead rings. The sponge can be viewed as a particular type of film (a sponge like film).

Silk fibroin solution (c=8.1% w/v) was used to mount the mesh onto the films. Specifically, 2 ml of silk solution were added to the lid of a sterile 100 cm Petri dish and were evenly spread with a sterile pipette tip. The mesh was placed in the dish until its surface was uniformly wet. The mesh was added onto the dried sponge (to the side that contacted the tray while freezing) then smoothed down with gloved fingers to ensure uniform surface attachment. Prototypes were dried for 1 hour, then placed in 90% ethanol for 30 minutes and blotted. Subsequently, the devices were placed in deionized water for 5 minutes for first wash. After the first wash, sponges were trimmed down to the size of the 6×6 cm mesh and then placed into second wash for 5 minutes. The devices were washed one more time then pouched.

Sterilization:

Devices were placed in metallized peelable polyester polyethylene film (MMPE) and paper polyethylene foil polyethylene barrier (PPFP) pouches, sealed using Accu-Seal Sealer Model 630, and e-beam sterilized.

Packaging/Storage:

Devices prepared as above were placed in PPFP pouches, sealed using Accu-Seal Sealer Model 630, and e-beam sterilized. Devices were then kept in pouches and stored under ambient conditions.

Testing:

Devices in both MMPE and PPFP pouches were examined two weeks after sterilization for overall integrity, delamination, pliability and suturability.

Observations:

The devices remained moist during and after sterilization, had good pliability and did not delaminate when crumpled in hand. The sponges did not shed when rubbed with gloved hands and the devices were easy to suture through.

### Device 10

Description:

SERI® Surgical Scaffold fused with SF/ALG sponge (6×6 cm)

Execution:

Non-sterile SERI® Surgical Scaffolds were cut with stainless steel scissors into 6×6 cm squares. Separately, silk fibroin solution (c=8.1% w/v) was mixed with ALG (c=2% w/v) in a 20:1 volume ratio then the mix was homogenized by pipetting up and down. To obtain a sponge-like biomaterial the solution (15 ml) was then cast in OmniTray lids and put into the −80° C. freezer for two hours. Frozen samples were lyophilized for 24 hours to dry, then treated with 15 ml of ethanol (100% v/v) for 45 min. Sponges were then removed from the tray, the edges were cut off, then were returned to the tray for an additional 30 minutes ethanol incubation. Subsequently, sponges were dried flat covered with OmniTray lids and lead rings. The sponge can be viewed as a particular type of film (a sponge like film).

Silk fibroin solution (c=8.1% w/v) was used to mount the mesh onto the films. Specifically, 2 ml of silk solution were added to the lid of a sterile 100 cm Petri dish and were evenly spread with a sterile pipette tip. The mesh was placed in the dish until its surface was uniformly wet. The mesh was added onto the dried sponge (to the side that contacted the tray while freezing) then smoothed down with gloved fingers to ensure uniform surface attachment. Prototypes were dried for 1 hour, then placed in 90% ethanol for 30 minutes and blotted. Subsequently, constructs were placed in deionized water for 5 minutes for first wash. After the first wash, sponges were trimmed down to the size of the 6×6 cm mesh and then placed into second wash for 5 minutes. The devices were washed one more time then pouched.

Sterilization:

Devices were placed in metallized peelable polyester polyethylene film (MMPE) and paper polyethylene foil polyethylene barrier (PPFP) pouches, sealed using Accu-Seal Sealer Model 630, and e-beam sterilized.

Packaging/Storage:

Devices prepared as above were placed in PPFP pouches, sealed using Accu-Seal Sealer Model 630, and e-beam sterilized. The devices were then kept in pouches and stored under ambient conditions.

Testing:

In both MMPE and PPFP pouches were examined two weeks after sterilization for overall integrity, delamination, pliability and suturability.

Observations:

Devices remained moist during and after sterilization. Devices appeared to have good pliability and did not delaminate when crumpled in hand. The sponges did not shed when rubbed with gloved hands. Devices were easy to suture through.

### Device 11

Description:

SERI® Surgical Scaffold fused with SF sponge (6×6 cm)

Execution:

Non-sterile SERI® Surgical Scaffolds were cut with sterile stainless steel scissors into 6×6 cm squares. Separately, to obtain a sponge-like biomaterial, silk fibroin solution (c=8.1% w/v) (7.5 ml) was cast in OmniTray lids and put into the −80° C. freezer for two hours. Frozen samples were lyophilized for 24 hours to dry, then treated with 15 ml of ethanol (100% v/v) for 45 min. Sponges were then removed from the tray, the edges were cut off, flipped over and returned to the tray for an additional 30 minutes of ethanol incubation. Subsequently, sponges were dried flat between 3 lint-free wipes, underneath a plastic tray with a lead ring on top. The sponge can be viewed as a particular type of film (a sponge like film).

Silk fibroin solution (c=8.1% w/v) was used to mount the mesh onto the films. Specifically, 2 ml of silk solution were added to the lid of a sterile 100 cm Petri dish and were evenly spread with a sterile pipette tip. The mesh was placed in the dish until its surface was uniformly wet Then the mesh was added onto the dried sponge (to the side that was in contact with the plate while freezing) and smoothed down with gloved fingers to ensure uniform surface attachment. Prototypes were allowed to dry flat under OmniTray lids for 1 hour. When dry, sponges were roughly trimmed, placed in 90% ethanol for 30 minutes, blotted, then put in deionized water for 5 minutes for first wash. After first wash, sponges were trimmed down to the size of the 6×6 cm mesh and then put into second wash for 5 minutes. The devices were then dried covered with OmniTrays and lead rings.

Sterilization:

Devices were placed in self-sealing pouches and ethylene oxide (EO) sterilized.

Packaging/Storage:

Devices prepared as above were placed in self-sealing sterilization pouches, were EO sterilized then aerated for at least 3 day prior use. During the aeration period, devices were kept under environmental conditions.

Testing:

Samples were visually assessed for integrity.

Observations:

Devices maintained their integrity during and after sterilization. No delamination, change in color or sponge cracking was notices upon visual inspection of the pouched devices.

### Device 11A

Description:

SERI® Surgical Scaffold fused with SF sponge (6×6 cm)

Execution:

Non-sterile SERI® Surgical Scaffolds were cut with stainless steel scissors into 6×6 cm squares. Separately, to obtain a sponge-like biomaterial, silk fibroin solution (c=8.1% w/v) (15 ml) was then cast in OmniTray lids and put into the −80° C. freezer for two hours. Frozen samples were lyophilized for 24 hours to dry, then treated with 15 ml of ethanol (100% v/v) for 45 min. Sponges were then removed from the tray, the edges were cut off, then were returned to the tray for an additional 30 minutes ethanol incubation. Subsequently, sponges were dried flat covered with OmniTray lids and lead rings. The sponge can be viewed as a particular type of film (a sponge like film).

Silk fibroin solution (c=8.1% w/v) was used to mount the mesh onto the films. Specifically, 2 ml of silk solution were added to the lid of a sterile 100 cm Petri dish and were evenly spread with a sterile pipette tip. The mesh was placed in the dish until its surface was uniformly wet. The mesh was added onto the dried sponge (to the side that contacted the tray while freezing) then smoothed down with gloved fingers to ensure uniform surface attachment. Prototypes were dried for 1 hour, then placed in 90% ethanol for 30 minutes and blotted. Subsequently, constructs were placed in deionized water for 5 minutes for first wash. After the first wash, sponges were trimmed down to the size of the 6×6 cm mesh and then placed into second wash for 5 minutes. The devices were washed one more time then pouched.

Sterilization:

Devices were placed in paper polyethylene foil polyethylene barrier (PPFP) pouches, sealed using Accu-Seal Sealer Model 630, and e-beam sterilized.

Packaging/Storage:

Devices prepared as above were placed in PPFP pouches, sealed using Accu-Seal Sealer Model 630, and e-beam sterilized. The devices were then kept in pouches and stored under ambient conditions.

Testing:

In both MMPE and PPFP pouches were examined two weeks after sterilization for overall integrity, delamination, pliability and suturability.

Observations:

Devices remained moist during and after sterilization. Devices appeared to have good pliability and did not delaminate when crumpled in hand. The sponges did not shed when rubbed with gloved hands. The devices were easy to suture through.

### Device Characterization

Materials
The devices made (as set forth above)
SERI® Surgical Scaffold (Allergan)
Dulbecco's Phosphate Buffered Saline 1× (DPBS) (ATCC, cat #30-2200)

Equipment
Thickness dial gauge (SNAP-004, Kafer J100 type C)
Mechanical testing equipment (Instron Model E3000)
Mechanical testing equipment (Instron Model 8871)

### Device Swelling

Swelling characterization is crucial for implantable devices since after surgical implantation of the device it is important that the device not cause tissue or nerve compression due to device volume increases. To determine the extent of any swelling of the anti-adhesive devices made, we compared dry device sample thicknesses to that of device samples incubated under physiological conditions. Thickness measurements were performed with a thickness dial gauge and 15 measurements were taken per 6×6 cm device. To mimic physiological environments, the devices were incubated in DPBS at 37° C., 50 rpm for 24 h. The results showed that devices 6, 7A, 8A, 9, 10 has no or an insignificant or a clearly unsubstantial amount of swelling (none or essentially no swelling at all, that is + or −5% of the reference value) (T TEST, p>0.05) when incubated under physiological conditions. The ORC prototypes (P1A, P2A and P3) were not included in this evaluation since ORC gels in the presence of water and would yield erroneous measurements.

### Mechanical Testing

The mechanical properties of the devices (devices 1A, 2A, 3, 6, 7A, 8A, 9, 10 and 11A) assessed their suitability for use in surgical soft tissue repair procedures. SERI® Surgical Scaffold (SERI mesh) was used as the reference material. Both tear testing and burst testing was carried out. Briefly, samples were cut into 40×40 mm for burst testing and 10×60 mm for tear testing and were immersed in PBS for 2 h at room temperature. For burst testing, samples were mounted on the specimen clamp and the ball burst fixture was pushed against the sample at a constant rate of 60 mm/in until sample failure. For tear testing, samples were affixed with clamps and pulled at a constant rate of 2400 mm/min until sample failure.

The burst strength results showed that the fusion of films or sponges to SERI® Surgical Scaffold did not improve and did or deteriorate the reference's intrinsic mechanical properties. All devices showed comparable to or very similar (that is + or −10% of the reference value) of to the burst strength values of the SERI® Surgical Scaffold control (t-test, p>0.05).

The tensile testing of devices 1A, 2A, 3, 6, 7A, 8A, 9, 10 and 11A) also showed close similarity to the control with comparable (i.e. + or −about 10% of the reference value) “Elongation at break: values obtained for all devices (t-test, p>0.05). However, device 10 was able to withstand about a 10% higher tensile loads at break as compared to the SERI reference control (t-test p=0.02).

Overall, this Example 4 showed that the swelling of the tested devices was negligible and they would not pose any risk of compression to the surrounding tissue post-implantation. The ORC materials could not be tested because the sacrificial layer gelled and disintegrated when hydrated, making sample manipulation impossible. Additionally, the mechanical properties of the devices tested were very similar to SERI® Surgical Scaffold showing that the devices can provide sufficient mechanical support when implanted to assist soft tissue repair.

### Example 5

### Single Layer and Two Layered Anti-Adhesive Surface Silk Medical Devices

This Example 5 discloses two types of silk medical devices we made and characterized. Both types of devices we made included a particular new, knitted silk mesh (or scaffold). The first type of device we made was a particular knitted silk mesh (or scaffold) prepared with at least one surface or side of the device having an anti-adhesive (i.e. having a smooth or low profile with full coverage of open space or pores) surface. This silk mesh of this first type of device bioresorbs after implantation over about 1-3 years. The second type of device we made also comprised a first layer of knitted silk mesh (scaffold), as with the first device, and with the anti-adhesive property provided by a sacrificial (second) layer attached to or fused to one side of the first knitted mesh layer of the second device. The sacrificial layer is comprised entirely or mainly of a faster (preferably over at least about 10 days and no more than about 30 days) bioresorbable yarn. Thus this two layer, second type of device has a front or top side made of the knitted silk mesh (which does not have an anti-adhesive property) and a back or bottom side formed by an anti-adhesive, sacrificial layer, which sacrificial layer can be made of quickly bioresorbing fibers, such as PGA, PLGA and/or ORC fibers. The anti-adhesive property of either the first type or device or of the second type of device prevents or reduces tissue (for example bowel tissue and/or abdominal viscera) adhesion to the (bottom or back) side of the device placed in contact with the bowel tissue or the abdominal viscera. It is important to note that the (top or front) side of the device is the knitted silk mesh layer of the device which top or front side of the device does not have an anti-adhesive property, and in fact the pores on the top or front side of the device (for both the first and the second type of device) facilitates tissue ingrowth onto and into the front or top side of the device. In this manner the device, as with the SERI® Surgical Scaffold, provides soft tissue mechanical support and a soft tissue load bearing function as new connective tissue forms onto and into the slowly biodegrading top or font side of the implanted device.

Thus both types of devices made in this Example 5 had an anti-adhesive property and additionally were made using a single step fabrication (textile knitting) process.

Thus we developed new silk based medical devices (with a knitted silk mesh layer) for use in various medical and surgical procedures, including in hernia repair procedures which silk based devices due to their anti-adhesive property can resist or prevent post-operative adhesion formations on the anti-adhesive side of the device.

### Materials

The four types of yarns we used for the embodiments of our invention made were:
6 filament, low twist, sericin extracted silk yarn.
9 filament, high twist, sericin extracted silk yarn.
45D PGA yarn (for example made by Teleflex Medical as Deknatel®). This is an18 filament, 45 denier, violet, polyglycolic acid (PGA) fiber processed with water-washable spin finish (up to 4% w/w).
128D PGA yarn (for example also made Teleflex Medical under the Deknatel® brand name). This is a 48 filament, 128 denier, undyed, polyglycolic acid (PGA) fiber processed with water-washable spin finish (up to 4% w/w).

The silk yarn was used to make the first type of device or to make the top of front side layer of the second type of device. The PGA (non-silk) yarns were used to make the sacrificial layer of the second type of device. A mineral oil (such as a heavy, white, mineral oil available from Avantor) was used to coat the yarns to facilitate their knitting. Oil residue can be later removed from the yarns and/or from the knitted device by a variety of methods including soaking (washing) and/or carbon dioxide treatment.

### Equipment

The backwinder (single head) used was made by SIMET as model number SE-01.

The knitting machine used was made by COMEZ as model number EL-800-8B. This is a double needle bed warp knitter with 8 bar capability including two long throw bars.

The thickness gauge made by Kafer as model number J-100 C

The testing equipment we used was made by Instron as model number E3000 (tensile tester).

As set forth above we made two types of silk based medical devices in this Example 5. All the devices made included at least a knitted silk mesh (scaffold), for example as the base layer. The first type of device made comprised only the knitted silk mesh with one side having a low profile, low sheer, full coverage, anti-adhesive property (i.e. satin knit). This first type of silk device was made to bioresorb over about 1-3 years after implantation. The second type of silk device we made comprised the knitted silk mesh layer of the first device attached or fused to a second, anti-adhesive, sacrificial knitted non-silk fiber layer. The sacrificial layer comprised entirely or mainly a faster (over at least about 10 days but over less than about 30 days) bioresorbable non-silk fibers.

Devices without a Sacrificial Layer (Base Layer Meshes)

For the base layer mesh of the devices we made with a sacrificial layer or with an anti-adhesive we developed a single layer using low denier, low twist yarn using a knit pattern that provides a low profile (smooth) surface to the material to thereby eliminate or minimize irritation to the bowel and hence remove or substantially reduce adhesion formation onto the side of the device (i.e. the smooth side facing the bowel tissue. Embodiment we made of such a suitable device we refer to as “the Single Bed 102” or as the “SBR 202”. The SBR 202 devices are six filament mesh devices.

Specifically, the Single Bed 102 devices were made as a series of “SS-P01-0X” devices, where X is an integer 1 and higher (i.e. the devices referenced as the series of devices P01-01-0X). Thus several versions of this device were made (i.e. the SS-P01-01-0X device versions). This device has a low profile (smooth) surface on the bowel facing side made on a single bed (front bed) knitting machine with about half the stitch density used for SERI Surgical Scaffold, resulting in a thinner, low sheer, full coverage knitted silk fabric and a low (smooth) loop profile.

Number of bars used: 03 (bars #4, 5, and 7)

Knitting beds: Front only (10 gauge).

Bed Spacing: 0.8 mm

Pick density: 18 picks/cm

Type of needle used: Latch needle (Comez part #61326, Groz-Beckert part # SN-S 51.60 G01)

Number of needles used: 25

Pattern length: 12

Creel setup:

Front creel: 28 ends on left side for bar #7 (lay-in)

25 ends on right side for bar #5 (pillar stitch).

Back creel: 25 ends on left side for bar #4 (pillar stitch)

Feed rollers setup:

Feeder #18: Feeding 25 ends to bar #4

Feeder #20: Feeding 02 ends to bar #7 (ends for outer most edges)

Feeder #21: Feeding 02 ends to bar #7 (second-in from outer edges)

Feeder #22: Feeding 25 ends to bar #5

Feeder #23: Feeding 24 ends to bar #7 (bulk of the lay-in)

Bar swing setup: 15.5 mm with centered swing

Chain links and bar threading

Bar #7 (lay-in) full set as 9-9, 9-9, 7-7, 7-7, 9-9, 9-9, 1-1, 1-1, 3-3, 3-3, 1-1, 1-1

Bar #5 (pillar) full set as (3-1, 1-1, 1-3, 3-3)×3

Bar #4 (pillar) full set as (1-3, 3-3, 3-1, 1-1)×3

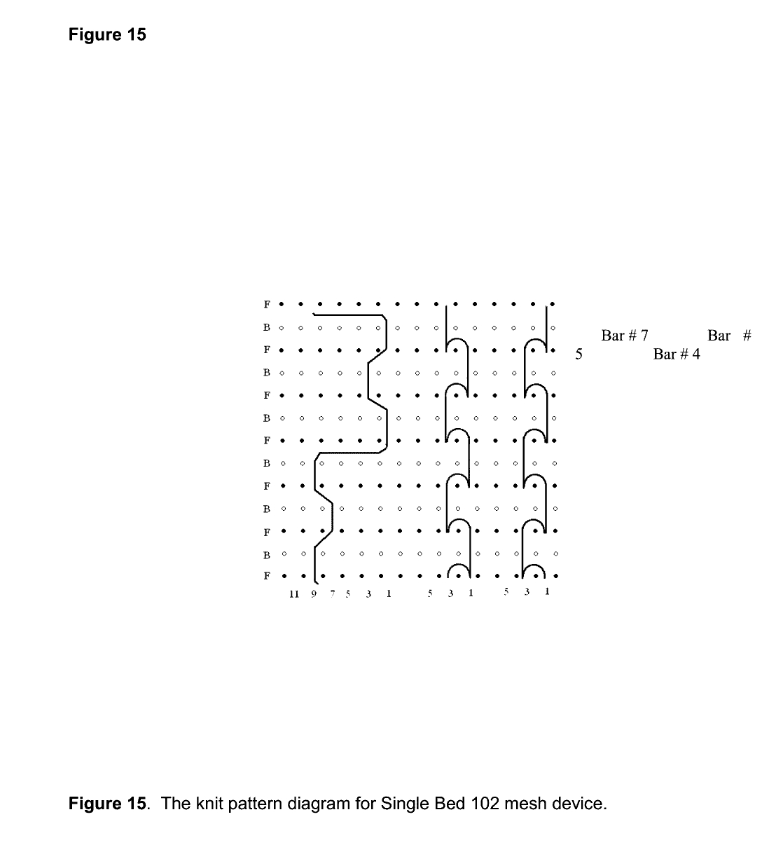

FIG. 15 shows the knit pattern diagram for Single Bed 102 mesh device and

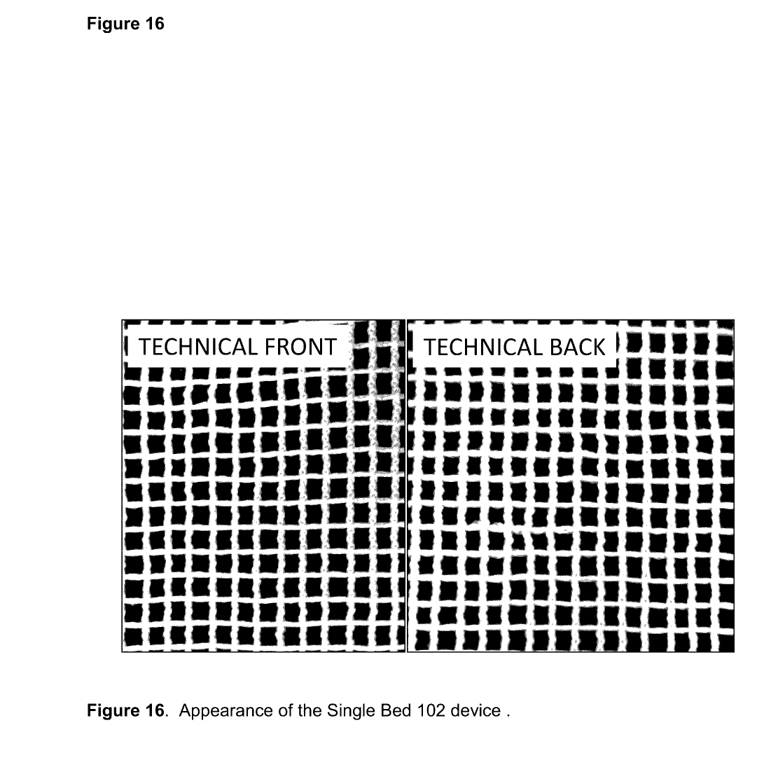

FIG. 16 shows the appearance of the Single Bed 102 device.

### Anti-Adhesive Knitted (Satin) Devices

With this device, the anti-adhesive layer (side facing bowel) was made using silk yarn and a satin knit pattern combined to the 55-P01-01 (single bed 102 design). The satin knitting consists of long strides of yarn crossing back and forth along more than one needle. This back and forth motion of the yarn creates a “wood stack” type of design that runs along the fabric course direction leading to a lustrous appearance and the “smooth” hand characteristic of satin fabrics. The percent coverage of the surface can be controlled by the crossing angle and the amount of yarn crossing at a given time. The crossing angle is controlled by the number of needles across which the yarn crosses and the amount of yarn is controlled by the number of threads per guide and the yarn denier (reference number satin series: SS-P02-02-0X).

Number of bars used: 04 (bars #2, 4, 5, and 7)

Knitting beds: Front only (10 gauge).

Bed Spacing: 1.0 mm

Pick density: 18 picks/cm

Type of needle used: Latch needle (Comez part #61326, Groz-Beckert part # SN-51.60 G01)

Number of needles used: 25 for S-P02-02-01 through 03

30 for S-P02-02-08 through 13

Pattern length: 12 for SS-P02-02-01, 02, 03, and 10

08 for SS-P02-02-13

04 for SS-P02-02-08, and 09

Creel setup: the settings below are for patterns made with 30 needles

Front creel: 33 ends on left side for bar #7 (lay-in)

30 ends on right side for bar #5 (pillar).

Back creel: 30 ends on left side for bar #4 (pillar)

N×(30−C) ends on left side for bar #2 (satin)

Where N is the number of ends/threads per guide. C is the number of needles that the satin yarn is crosses in each stride.

Feed rollers setup:

Feeder #17: Feeding N×(30−C) ends to bar #2

Feeder #18: Feeding 30 ends to bar #4

Feeder #20: Feeding 02 ends to bar #7 (ends for outer most edges)

Feeder #21: Feeding 02 ends to bar #7 (second-in from outer edges)

Feeder #22: Feeding 30 ends to bar #5

Feeder #23: Feeding 29 ends to bar #7 (bulk of the lay-in)

Bar swing setup: 15.5 mm with centered swing.

Bar #7 (lay-in) full threading: 9-9, 9-9, 7-7, 7-7, 9-9, 9-9, 1-1, 1-1, 3-3, 3-3, 1-1, 1-1

Bar #5 (pillar) full threading: (3-1, 1-1, 1-3, 3-3)×3

Bar #4 (pillar) full threading: (1-3, 3-3, 3-1, 1-1)×3

Bar #2 (satin) full threading: (3-1, 5-5, 9-11, 5-5)×3

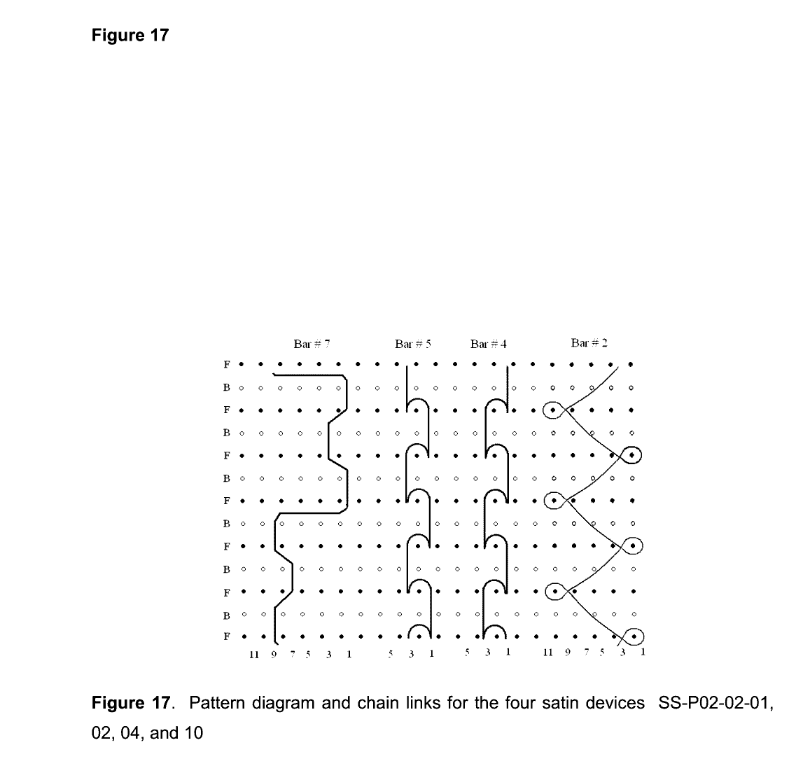

FIG. 17 shows the knit pattern diagram for certain satin devices and

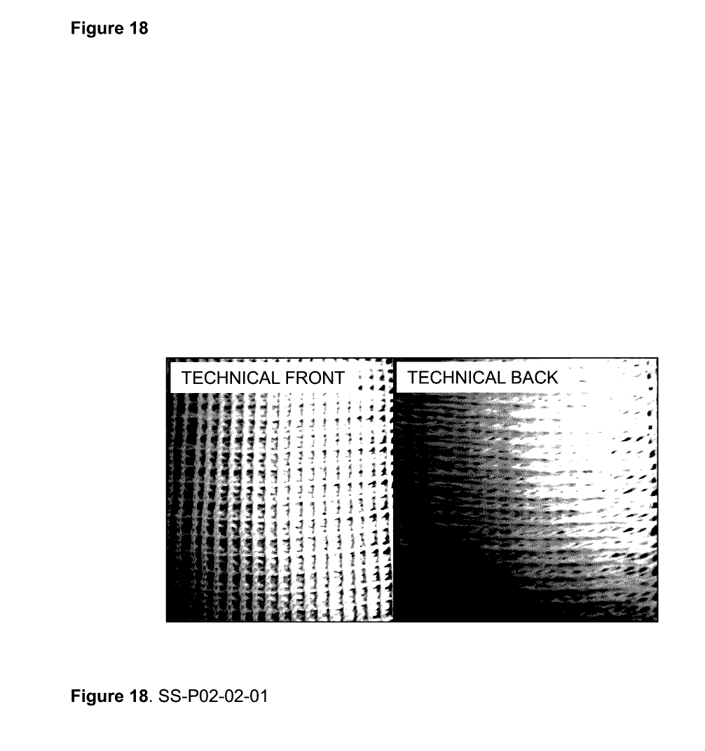

FIG. 18 shows the appearance of a silk based satin device.

Devices with a Sacrificial Layer

The second type of devices we made in this Example 5 had a sacrificial layer. The (top or front) side facing the abdominal wall or muscle area was made primarily or entirely of knitted silk (that is porous and mechanically strong) and promotes tissue integration. The (bottom or back) side facing the bowel comprises the sacrificial layer. This bottom or back side layer is made of a material or a composite that allows temporary tissue adhesion to the sacrificial layer to occur. Shortly after implantation (within about 10 days to about 30 days after implantation), the sacrificial layer is mechanically compromised by being biodegraded and bioresorbed leading to the separation of the adhering tissue (i.e. bowel tissue) from the device. The sacrificial layer comprising devices are biocompatible, made by knitting (textile machinery) of yarns by a twisting, backwinding, and warp knitting process, and as noted can for example lose at least 50% of their mechanical integrity or strength within 10-30 days after implantation of the device with such a sacrificial layer. We determined that suitable materials to comprise the sacrificial layer can be knittable non-silk fibers of polyglycolic acid (PGA), poly lactic-co-glycolic acid (PLGA), oxidized regenerated cellulose (ORC), carboxymethylcellulose (CMC) and combinations thereof. The sacrificial layers of embodiment made were made using the 45D PGA yarn but can also be made using various deniers of PGA, PLGA, ORC, CMC or a combination thereof.

Embodiments of Sacrificial Layer comprising devices made:

1. Shag carpet device (several versions of this shag carpet device were made, as the S-P02-02-0X and SS-P02-03-0X device versions). These devices had a shag carpet like structure with the protruding loops act as the sacrificial layer (side facing bowel). The shag carpet devices consisted of two components or layers. The first was the base layer of (knitted silk) fabric that provided the overall fabric integrity and the load distribution when subjected to external mechanical forces. The second layer was a loose (non-silk) knitted yarn that forms extended loops protruding vertically away from the base fabric plane, hence giving the fabric its characteristic loopy or shag like texture. The percent loop coverage of the surface can be controlled by the loop length (controlled by feed rate), the amount of loose yarn per loop (controlled both by feed rate, yarn count, and number of threads per count), and the number of loops per surface area (controlled by machine gauge used and pattern).

In the case of the devices described, loops can be formed on the base silk fabric (device SS-P01-01) by using a simple closed (or open) tricot stitch swinging back and forth between adjacent needles with high feed rate.

Number of bars used: 04 (bars #2, 4, 5, and 7)

Knitting beds: Front only (10 gauge)

Bed Spacing: 1.0 mm

Pick density: 18 picks/cm

Type of needle used: Latch needle (Comez part #61326, Groz-Beckert part # SN-S 51.60 G01)

Number of needles used: 25 for SS-P02-02-05 and 06 and SS-P02-03-02 through 09

30 for SS-P02-02-06, 07, and 12

Pattern length: 04 for SS-P02-02-12 and 12 for other than SS-P02-02-12

Creel setup: the settings below are for the patterns made with 30 needles

Front creel: 33 ends on left side for bar #7 (lay-in)

30 ends on right side for bar #5 (pillar).

Back creel: 30 ends on left side for bar #4 (pillar)

29 ends on left side for bar #2 (loops)

Feed rollers setup:

Feeder #17: Feeding 29 ends to bar #2 (loops)

Feeder #18: Feeding 30 ends to bar #4 (pillar)

Feeder #20: Feeding 02 ends to bar #7 (ends for outer most edges)

Feeder #21: Feeding 02 ends to bar #7 (second-in from outer edges)

Feeder #22: Feeding 30 ends to bar #5 (pillar)

Feeder #23: Feeding 29 ends to bar #7 (bulk of the lay-in)

Bar swing setup: 15.5 mm with centered swing

The pattern for all ‘shag carpet’ devices except for SS-P02-02-12 was;

Bar #7 (lay-in) full threading as 9-9, 9-9, 7-7, 7-7, 9-9, 9-9, 1-1, 1-1, 3-3, 3-3, 1-1, 1-1

Bar #5 (pillar) full threading as (3-1, 1-1, 1-3, 3-3)×3

Bar #4 (pillar) full threading as (1-3, 3-3, 3-1, 1-1)×3

Bar #2 (satin) full threading as (3-1, 3-3, 3-5, 3-3)×3

The Pattern for device SS-P02-02-12 was:

Bar #7 (lay-in) full threading as 9-9, 9-9, 1-1, 1-1

Bar #5 (pillar) full threading as 3-1, 1-1, 1-3, 3-3

Bar #4 (pillar) full threading as 1-3, 3-3, 3-1, 1-1

Bar #2 (satin) full threading as 3-1, 3-3, 3-5, 3-3

Guide (heddle) threading: For all prototypes, Bars #4, 5, and 7 were single threaded (one end per heddle). As for bar #2, the following threading was used:

Single for SS-P02-03-02 through 09 and SS-P02-02-05

Double for SS-P02-02-06

Triple 45D PGA violet for SS-P02-02-07

Triple 45D PGA violet for SS-P02-02-12.

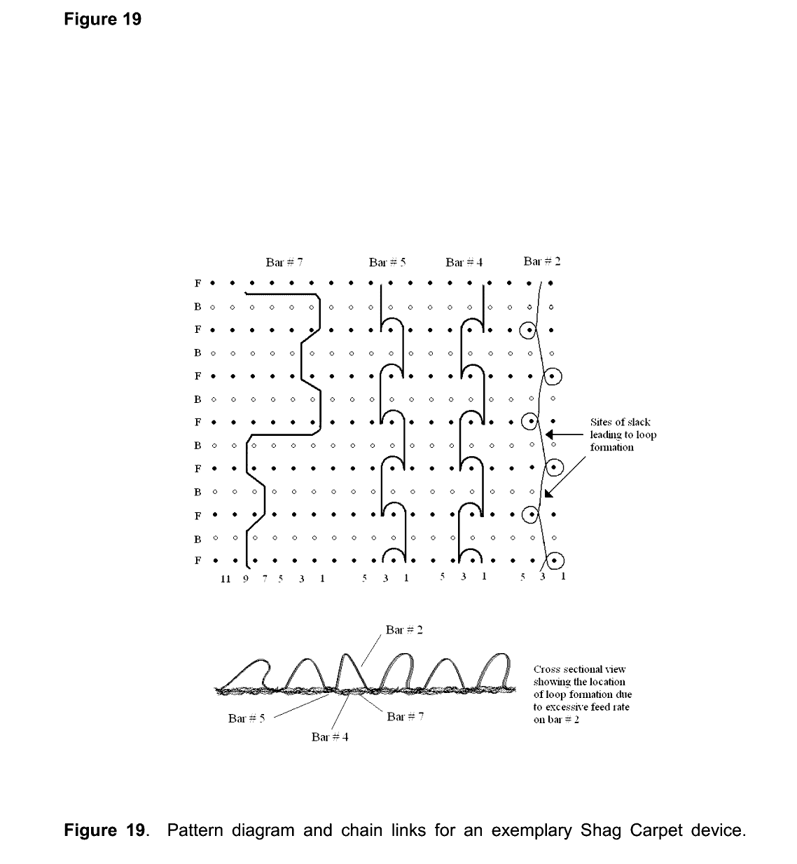

FIG. 19 shows a pattern diagram and chain links for an exemplary Shag Carpet device.

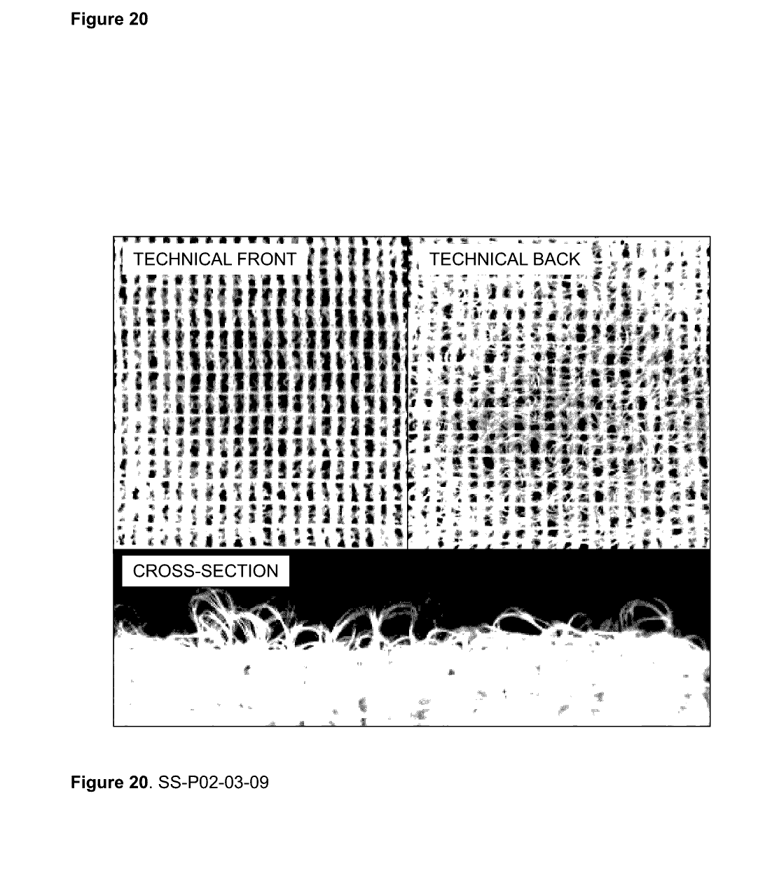

FIG. 20 shows the appearance of an exemplary knitted fabric (shag carpet) device.

2. Another embodiment of a sacrificial layer comprising device made was the satin series reference number SS-P02-02-0X. With these devices, the sacrificial, non-silk layer (the side facing the bowel, and made of PGA, PLGA, ORC, CMC or combinations thereof) was made using a ‘satin’ knit pattern combined to the SS-P01-01 (single bed 102 design). ‘Satin’ consists of long strides of non-silk yarn crossing back and forth along more than one needle. This back and forth motion of the yarn creates ‘wood stack’ type of design that run along the fabric course direction leading to a lustrous appearance and smooth hand characteristic of ‘satin’ fabrics. The percent coverage of the surface can be controlled by the crossing angle and the amount of yarn crossing at a given time. The crossing angle is controlled by the number of needles across which the yarn crosses and the amount of yarn is controlled by the number of threads per guide and the yarn denier.

Number of bars used: 04 (bars #2, 4, 5, and 7)

Knitting beds: Front only (10 gauge).

Bed Spacing: 1.0 mm

Pick density: 18 picks/cm

Type of needle used: Latch needle (Comez part #61326, Groz-Beckert part # SN-51.60 G01)

Number of needles used: 25 for S-P02-02-01 through 03

30 for S-P02-02-08 through 13

Pattern length: 12 for SS-P02-02-01, 02, 03, and 10

08 for SS-P02-02-13

04 for SS-P02-02-08, and 09

Creel setup: the settings below are for patterns made with 30 needles

Front creel: 33 ends on left side for bar #7 (lay-in)

30 ends on right side for bar #5 (pillar).

Back creel: 30 ends on left side for bar #4 (pillar)

N×(30−C) ends on left side for bar #2 (satin)

Where N is the number of ends/threads per guide. C is the number of needles that the ‘satin’ yarn is crosses in each stride.

Feed rollers setup:

Feeder #17: Feeding N×(30−C) ends to bar #2

Feeder #18: Feeding 30 ends to bar #4

Feeder #20: Feeding 02 ends to bar #7 (ends for outer most edges)

Feeder #21: Feeding 02 ends to bar #7 (second-in from outer edges)

Feeder #22: Feeding 30 ends to bar #5

Feeder #23: Feeding 29 ends to bar #7 (bulk of the lay-in)

Bar swing setup: 15.5 mm with centered swing.

Bar #7 (lay-in) full threading: 9-9, 9-9, 7-7, 7-7, 9-9, 9-9, 1-1, 1-1, 3-3, 3-3, 1-1, 1-1

Bar #5 (pillar) full threading: (3-1, 1-1, 1-3, 3-3)×3

Bar #4 (pillar) full threading: (1-3, 3-3, 3-1, 1-1)×3

Bar #2 (satin) full threading: (3-1, 5-5, 9-11, 5-5)×3

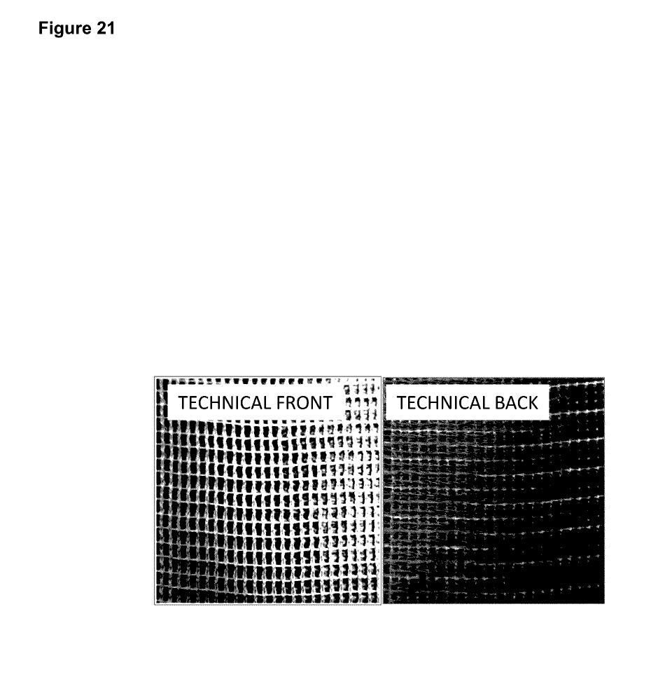

FIG. 21 shows the appearance of a representative sacrificial layer comprising satin device.

Devices with Detachable Layers (several versions of this device were made, as the SS-P04-0X detachable layer version series)

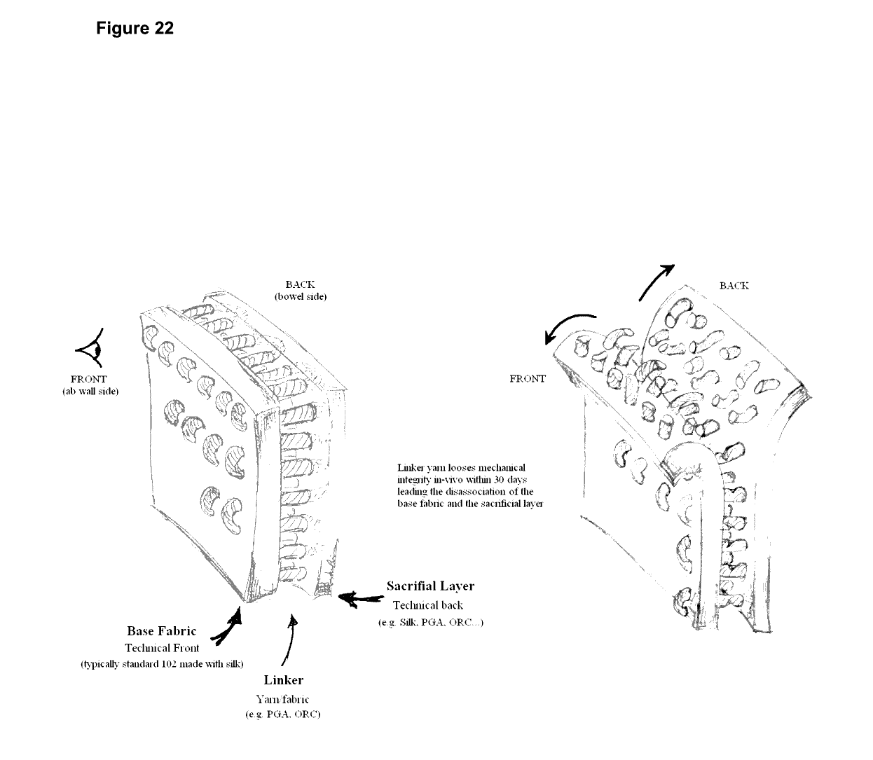

Unlike the satin and the shag carpet devices, the non-silk sacrificial layer in this device was not integrated with the base knitted silk fabric. It instead constituted an independent layer that peeled away from the base fabric within 30 days of implantation. It was typically a tightly knit non-silk fabric with small pore size (70-200 micron diameter). As depicted in FIG. 22. The non-silk sacrificial layer and the knitted silk base fabric were linked together using a fast resorbing/degrading yarn that was designed to be the first component of the assemble to fail mechanically leading therefore to the separation of the two layers.

To do so, a double needle bed was needed. The front bed was used to knit the base fabric as in SS-P01-01 (bars #4, 5, and 7) made out of silk and integrates with the abdominal wall tissue and act as the main load carrier. Meanwhile, the back bed was used to knit the sacrificial layer (bar #1). This layer can be made using either slow bioresorbing material like silk or fast resorbing material like PGA or ORC. Finally, the yarn used to link both fabrics (layers) was threaded through a dedicated bar (bar #2) and knits on both beds. This yarn consisted of low denier PGA or any other fast resorbing/degrading yarn, e.g. PLGA (90-10) and ORC yarns.

The detachable (triple) layered devices (reference number of detachable layered devices: SS-P04-0X), unlike for satin and “shag carpet” devices, these embodiments knit independently two textile layers (the base layer and the detaching layer) and then knit them together with a fast resorbing/degrading yarn (resulting in a three layered device). The middle layer disintegrated and so separated of the two outer layers within 10-30 days of implantation (see FIG. 21), thus preventing the adhesion of tissue to the base layer while keeping the tissue covered with the detaching layer. The detaching layer consisted of a tightly knit non-silk fabric with small pore size (70-200 micron diameter). A double needle bed knitter was used. The front bed was used to knit the base fabric as in SS-P01-01 (bars #4, 5, and 7). Meanwhile, the back bed was used to knit the detaching layer (bar #1). This layer can be made using either slow bioresorbing material like silk or fast resorbing material like PGA, PLGA or ORC. Finally, the yarn used to link both fabrics (layers) was threaded through a dedicated bar (bar #2) and knits on both beds (constituting the middle layer). This yarn consisted of low denier PGA or any other fast resorbing/degrading yarn, e.g. PLGA (90-10) and ORC yarns.

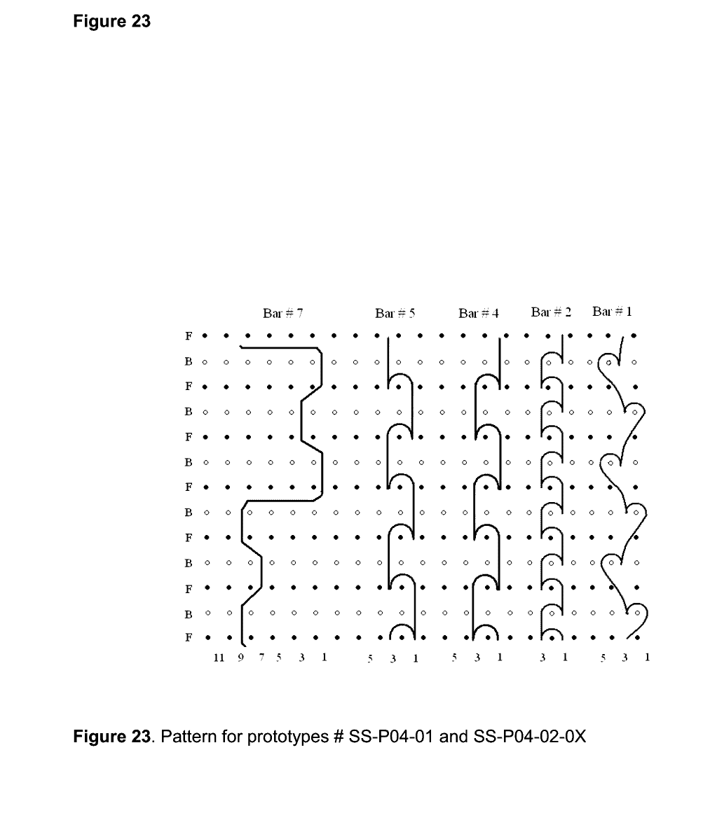

The percent coverage of the surface was controlled by the gauge used on the back bed, crossing angle, and the amount of yarn crossing at a given time. The crossing angle was controlled by the number of needles across which the yarn crosses and the amount of yarn is controlled by the number of threads per guide and the yarn denier. Weft insertion between both fabric layers was an additional option to increase surface coverage and shield direct exposure between silk (in the base fabric) and bowel surface. FIG. 22 illustrates this three layer device and FIG. 23 shows the pattern diagram and chain links used for an embodiment of such a device.

Number of bars used: 05 (bars #1, 2, 4, 5, and 7)

Weft insertion: none for SS-P04-01

Single 45D PGA for SS-P04-02-01 and 03

Triple 45D PGA for SS-P04-02-02

Note: the weft insertion bar was place at the bar position #3

Knitting beds: Front (10 gauge) and back (20 gauge) beds.

Bed spacing: 1.0 mm

Pick density: 22 picks/cm

Type of needle used: Latch needle (Comez part #61326, Groz-Beckert part # SN-51.60 G01)

Number of needles used: 30

Pattern length: 12

Creel setup: the settings was for patterns made with 30 needles

Front creel: 33 ends on left side for bar #7 (lay-in—base fabric)

30 ends on right side for bar #5 (pillar—base fabric)

Back creel: 30 ends on left side for bar #4 (pillar—base fabric)

30 ends on right side for bar #2 (pillar—linker)

60 ends on right side for bar #1 (tricot—sacrificial fabric)

1-4 ends in the middle for weft insertion

Feed rollers setup: Feeder #16: Feeding 60 ends to bar #1

Feeder #17: Feeding 30 ends to bar #2

Feeder #18: Feeding 30 ends to bar #4

Feeder #20: Feeding 02 ends to bar #7 (ends for outer most edges)

Feeder #21: Feeding 02 ends to bar #7 (second-in from outer edges)

Feeder #22: Feeding 30 ends to bar #5

Feeder #23: Feeding 29 ends to bar #7 (bulk of the lay-in)

Bar swing setup: 3.5 mm with centered swing

Bar #7 (lay-in) full 10 gg threading: 9-9, 9-9, 7-7, 7-7, 9-9, 9-9, 1-1, 1-1, 3-3, 3-3, 1-1, 1-1

Bar #5 (pillar) full 10 gg threading: (3-1, 1-1, 1-3, 3-3)×3

Bar #4 (pillar) full 10 gg threading: (1-3, 3-3, 3-1, 1-1)×3

Bar #2 (linker) full 10 gg threading: (1-3, 3-1)×6

Bar #2 (tricot) full 20 gg threading: (3-3, 1-3, 3-3, 5-3)×3

Note: Variants of the ‘detachable layer concept can be made by changing the chain links on bar #1 from closed to open loops and by varying the number of needled (C) crossed per swing.

Guide (heddle) threading: For all prototypes, Bars #4, 5, and 7 were single threaded (one end per heddle) using SUB-YN09E-001.

As for bar #2 the following threading was used:

Single for SS-P04-01

Single 45D PGA violet for SS-P04-02-0X

As for bar #1 the following threading was used:

Single for SS-P04-03

Double 45D PGA violet for SS-P04-02-01 and 02.

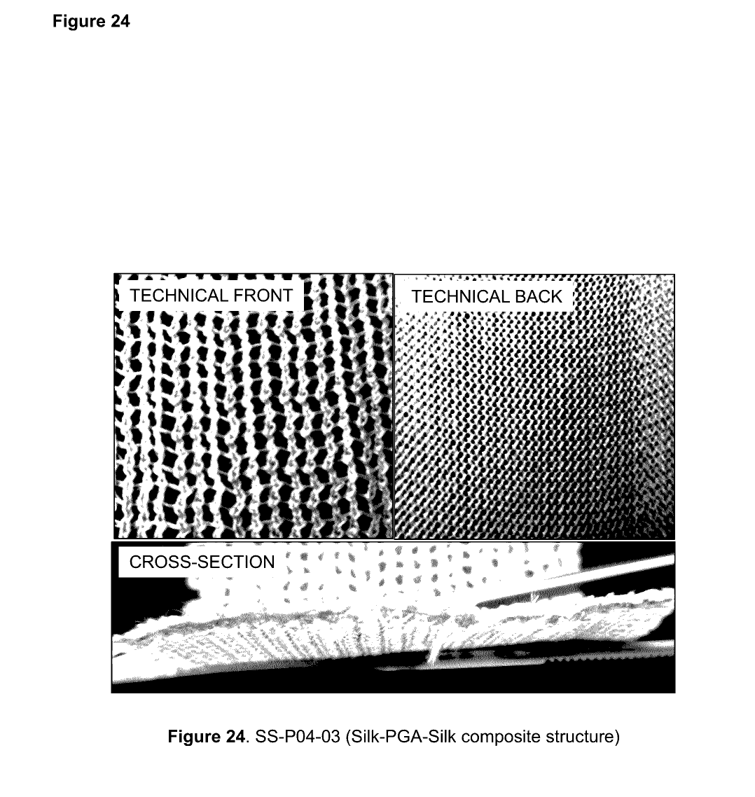

FIG. 24 shows the appearance of the knitted fabric

### Device Testing and Characterization

All device testing was performed with a sample size of n=15.

### Thickness

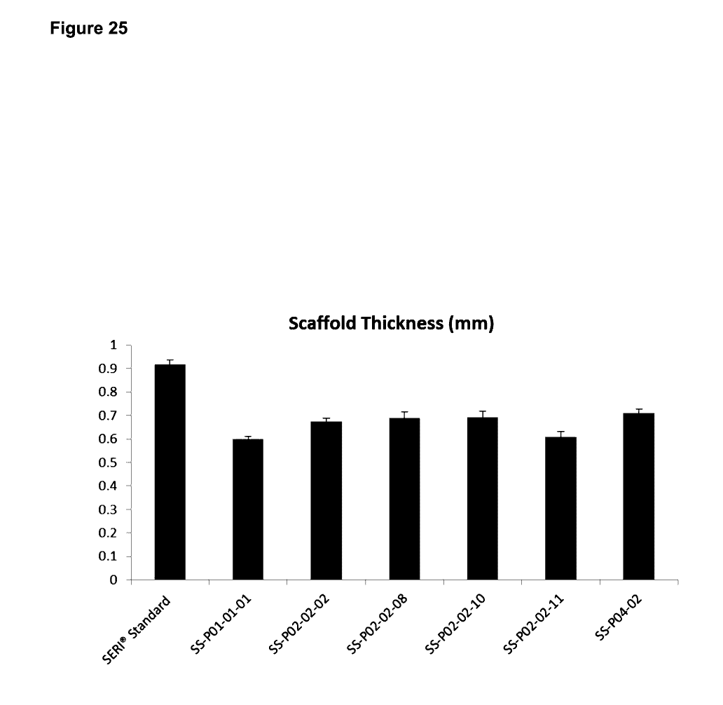

Device (n=15) thickness was measured using the J-100 Kafer thickness gauge. The average thickness values±one standard deviation are shown in the FIG. 25 graph below. All SS-P0X devices were 22 to 37% percent thinner than SERI Surgical Scaffold (also known as Standard 102 or as SERI® Standard). This confirms the thinner profile produced from knitting on a single bed with low pick density. The thinner profile along with the non-looping course design resulted in a smoother fabric (hand feel). SS-P01-01-01 had the lowest thickness value that correlates with the absence of any added material (e.g. PGA, ORC, etc. to the back of the fabric.

### Burst Strength

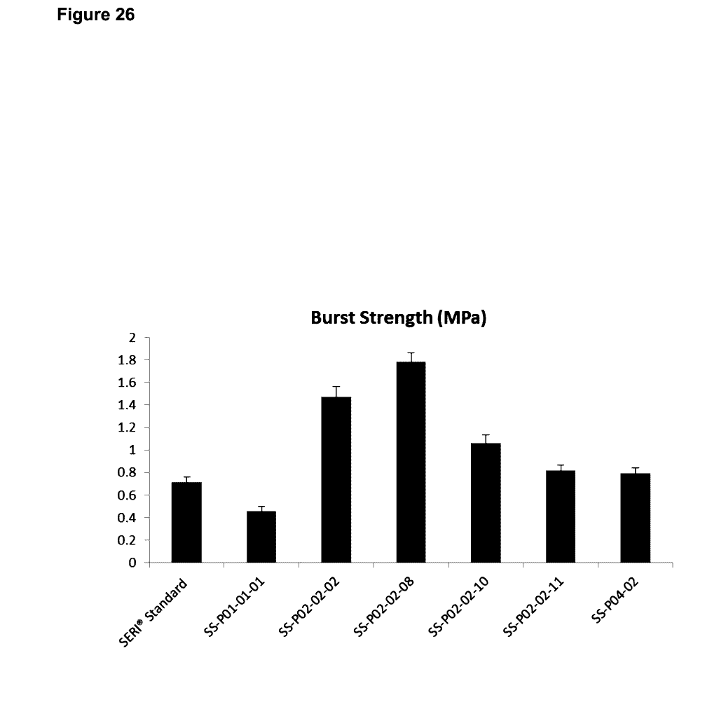

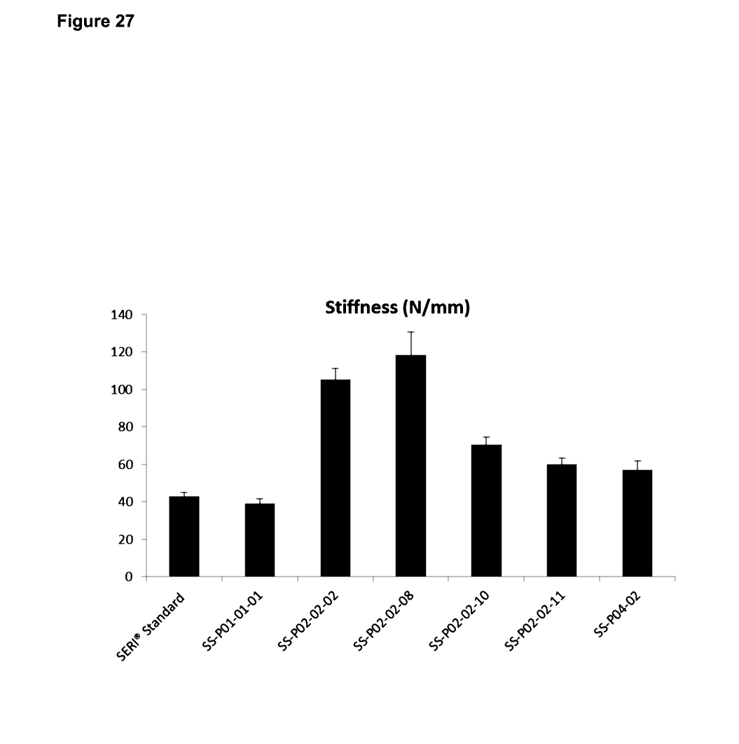

Device burst testing was carried out. Average burst strength and stiffness values±one standard deviation are shown in FIGS. 26 and 27.

Except for 55-P01-01-01, all other tested SS-PDX series had 10% to 150% increase in the burst strength and stiffness at time 0 when compared to the SERI® Standard. The lower values recorded for 55-P01-01-01 can be explained by the lower densities of silk used to make the prototype and lack of any other yarns attached to the back, i.e. PGA.

For abdominal wall reconstruction, the maximal anatomical (intra-abdominal) pressure is about 20 kPa. To withstand this, the device has a minimal burst strength of about 0.11 MPa Based on these considerations, the burst strength values determined for the silk/PGA prototypes indicated that they were suitable for abdominal reconstruction procedures. In terms burst stiffness, any device with a stiffness value equal or higher than the SERI® Standard can be suitable for abdominal wall reconstruction.

### Suture Pull Out

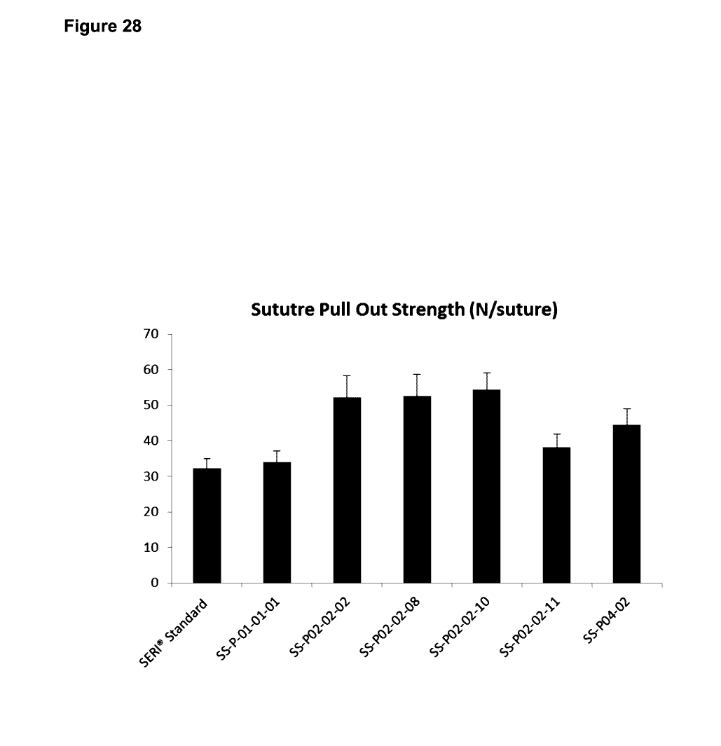

Device suture pull out testing was carried out. Average suture pull-out strength values±one standard deviation are reported in FIG. 28.

The suture pull-out strength for all SS-PDX prototypes was equal or higher (up to 70% higher in the case of SS-P02-02-10) to that of the SERI® Standard at time zero. Given that the SERI® Standard suture pull-out strength is compatible with abdominal wall repair procedures, all SS-PDX prototypes can perform adequately in the abdominal setting.

### Tensile Testing

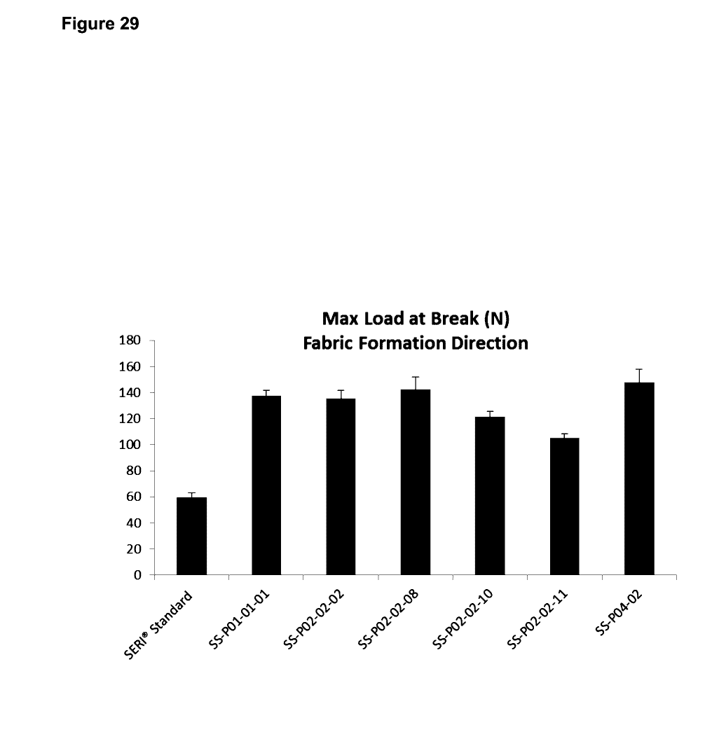

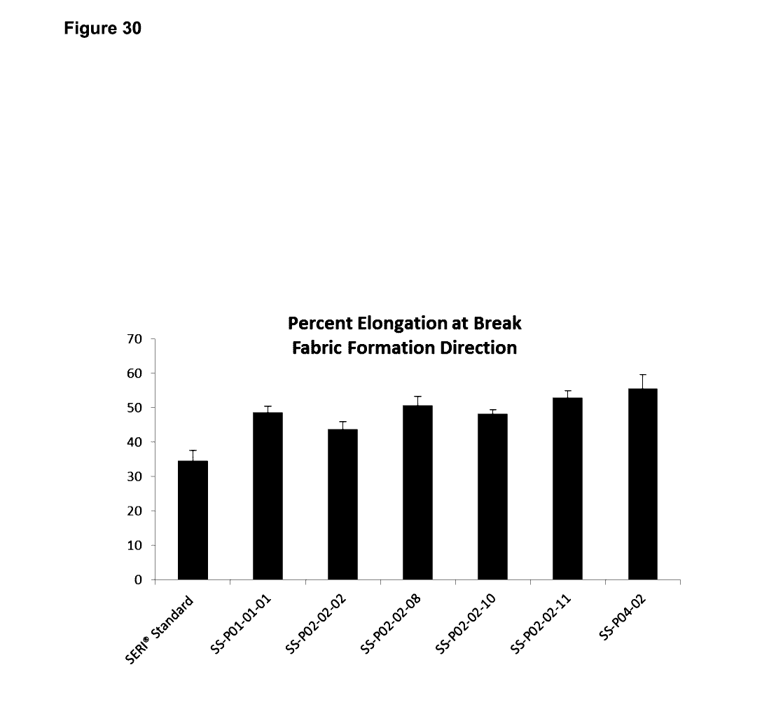

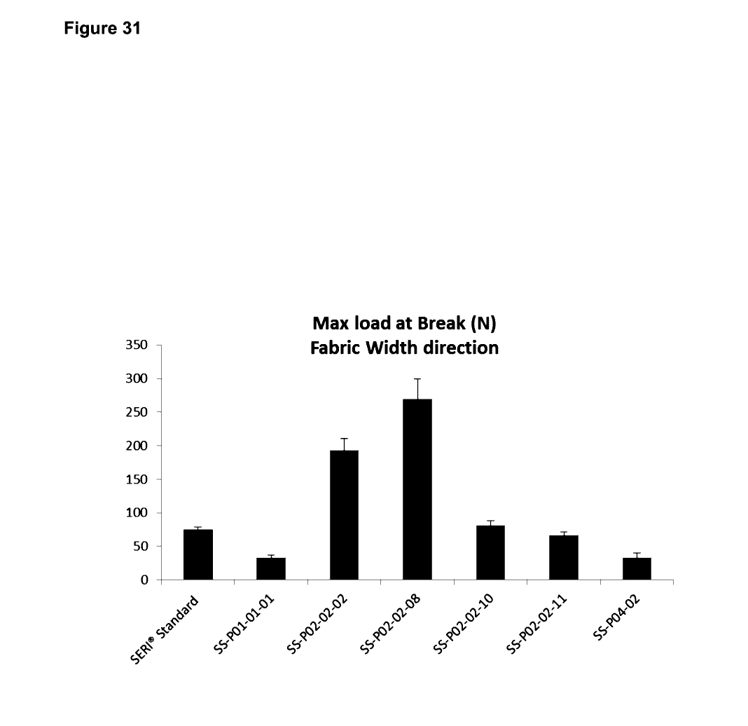

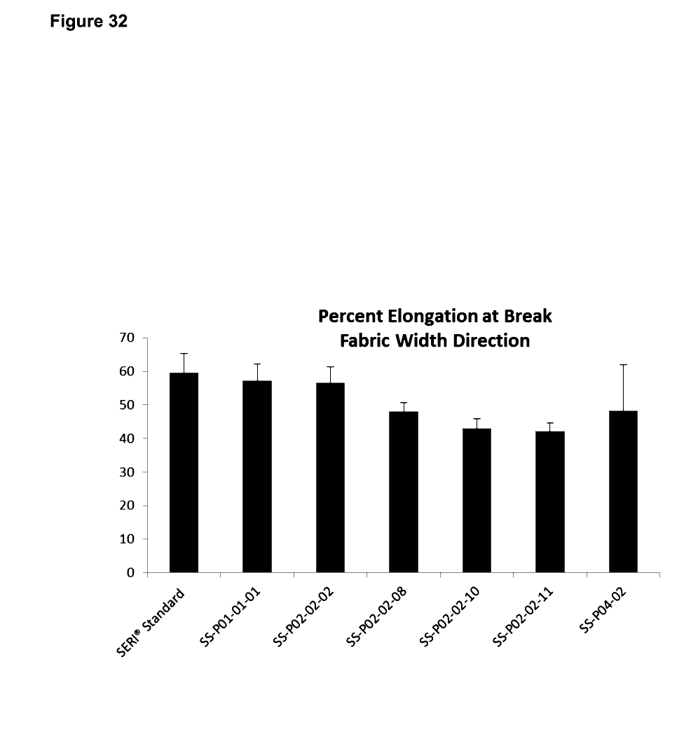

Device tensile testing was performed both in the wale and course directions. Tensile testing (single pull to failure) was performed in the direction of fabric formation and in the fabric width (course) direction. Average tensile strength, % elongation at break, and values±one standard deviation are reported in FIGS. 29 to 32. FIG. 29 shows the maximum load in the machine (fabric length) direction. FIG. 30 shows the percent elongation at break in the machine (fabric length) direction. FIG. 31 shows the maximum load in the course (fabric width) direction. FIG. 32 shows the percent elongation at break in the course (fabric width) direction.

Based on these results, all SS-PDX were 77-150% stronger in the machine direction then the SERI® Standard. Along the fabric width however, the strength was ranging from half as strong (SS-P01-01-01) to two and half times stronger (SS-P02-02-08) than SERI® Standard. Additionally, the pattern change described for the satin devices, that decrease the device pore size 3×, lead to a threefold increase in the strength along the fabric width. This is a result of a threefold increase of courses per unit length (SS-P02-02-02 and SS-P02-02-08). These results illustrate how the mechanical properties of the knitted devices can be modulated by controlling the knit design.

In closing, it is to be understood that although aspects of the present specification have been described with reference to the various embodiments, one skilled in the art will readily appreciate that the specific examples disclosed are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing quantities of ingredients, properties such as molecular weight, reaction conditions, and so forth used in the specification and claims are to be understood as being modified in all instances by the term “about.” As used herein, the term “about” means that the item, parameter or term so qualified encompasses a range of plus or minus ten percent above and below the value of the stated item, parameter or term. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A laminate, implantable silk medical device comprising:
(a) a first base layer comprising a knitted silk fabric, the first later having a top side and a bottom side, and;
(b) a second layer comprising a silk film or sponge fused to at least a portion of the bottom side of the first layer, thereby obtaining a laminate, implantable silk medical device.

2. The medical device of claim 1, wherein the silk film is water resistant.

3. The medical device of claim 1, wherein the silk film or sponge is fused to the silk fabric by drying the silk film or sponge after placing the silk film onto the silk fabric.

4. A process for making a laminate, implantable silk medical device, the process comprising:
(a) knitting a fabric from sericin depleted silk thereby making a first layer having a top side and a bottom side, and;
(b) preparing a silk solution by dissolving silk into a solvent;
(c) casting a silk film or sponge from the silk solution;
(d) treating the silk film or sponge so that at least one side of the silk film is water resistant, thereby forming a second layer; and
(e) fusing the second layer to at least a portion of the bottom side of the first layer, thereby obtaining a laminate, implantable silk medical device.

5. A method for providing tissue support and reducing adhesion formation, the method comprising the steps of implanting the device of claim 1.

6. An abdominal surgical method comprising the step of implanting the device of claim 1.

7. A laminate, implantable silk medical device comprising:
(a) a first layer comprising a water resistant, non-adherent silk film or sponge, the first layer having a top side and a bottom side, and;
(b) a second layer comprising a water soluble, adherent silk film or sponge formed on or placed on the top side of the first layer, thereby obtaining a laminate, implantable silk medical device.

8. The device of claim 7, wherein the silk film or sponge comprises silk and a compound selected from the group consisting of polyethylene glycol, ethylene oxide, propylene oxide block copolymer, hyaluronic acid, dextran, and alginate and salts and combinations thereof.
